(19) United States
(12) Patent Application Publication
(10) Pub. No.: US 2026/0289159 A1
(43) Pub. Date: Sep. 24, 2026

(54) SYSTEM

(71) Applicant: SoftBank Group Corp., Tokyo (JP)

(72) Inventor: Kenta HOKI, Tokyo (JP)

(73) Assignee: SoftBank Group Corp., Tokyo (JP)

(21) Appl. No.: 19/558,565

(22) Filed: Mar. 6, 2026

(30) Foreign Application Priority Data
Mar. 19, 2025 (JP) 2025-044503

(51) Int. Cl. G06F 40/40; G06F 40/284; G06N 3/0475; G16H 70/60

(52) U.S. Cl. CPC G06F 40/40; G06F 40/284; G06N 3/0475; G16H 70/60

(57) ABSTRACT

A system includes a processor that is configured to provide a user interface that receives input of information including a cause, symptoms, and elapsed time, generate a prompt sentence that instructs a generative artificial intelligence model to perform an analysis based on the input information, input the generated prompt sentence into the generative artificial intelligence model and obtain an analysis result from the generative artificial intelligence model, and calculate a degree of urgency for visiting a medical institution based on the analysis result and display the calculated degree of urgency on the user interface.

### CROSS-REFERENCE TO RELATED APPLICATION

This application is based on and claims priority under 35 USC 119 from Japanese Patent Application No. 2025-044503 filed on Mar. 19, 2025, the disclosure of which is incorporated by reference herein.

### BACKGROUND

### Technical Field

The present disclosure relates to a system.

### Related Art

Japanese Patent Application Laid-Open (JP-A) No. 2022-180282 discloses a persona chatbot control method executed by at least one processor. The method includes steps of: receiving a user utterance, adding the user utterance to a prompt including a description of a chatbot character and an associated instruction sentence, encoding the prompt, and inputting the encoded prompt to a language model to generate a chatbot utterance responding to the user utterance.

In conventional medical triage support systems, information entered by a user, such as cause, symptoms, and elapsed time, is often processed by fixed rule-based logic or static algorithms. Such systems have limited flexibility in handling diverse and complex combinations of symptoms, may provide inaccurate or insufficiently individualized urgency assessments, and are difficult to update rapidly in response to newly accumulated case data. In addition, conventional systems frequently lack an integrated mechanism for utilizing user feedback to continuously improve the accuracy of triage, and they may not automatically support communication with emergency responders when a severe condition requiring immediate medical attention is detected. As a result, there is a need for a system that can (i) flexibly analyze user-entered medical information using a generative artificial intelligence model, (ii) compute and present an appropriate degree of urgency for visiting a medical institution, (iii) improve its accuracy over time by analyzing user feedback through the same generative artificial intelligence model, and (iv) automatically generate and transmit appropriate contact content to emergency responders when an emergency condition is estimated.

### SUMMARY

In order to solve the above-described problems, an embodiment of the present invention provides a system comprising a processor, wherein the processor is configured to provide a user interface that receives input of information including a cause, symptoms, and elapsed time. The processor is further configured to generate a prompt sentence that instructs a generative artificial intelligence model to perform an analysis based on the input information, to input the generated prompt sentence into the generative artificial intelligence model, and to obtain an analysis result from the generative artificial intelligence model. The processor is configured to calculate a degree of urgency for visiting a medical institution based on the analysis result and to display the calculated degree of urgency on the user interface. The processor is further configured to receive feedback from a user, to generate a prompt sentence that instructs the generative artificial intelligence model to perform an analysis of the feedback, and to analyze the feedback using the generative artificial intelligence model in order to improve accuracy of the system. Additionally, the processor is configured, when it is estimated that a condition is severe and requires an immediate visit to a medical institution, to generate a prompt sentence that instructs the generative artificial intelligence model to generate contact content for an emergency responder, and to automatically transmit the contact content to the emergency responder.

The term “system” refers to an apparatus or a combination of one or more devices including at least one processor and, optionally, memory, communication interfaces, and user interface components configured to execute the functions described in the claims.

The term “processor” refers to any hardware component or combination of hardware components capable of executing instructions, including but not limited to a central processing unit (CPU), a microcontroller, a digital signal processor (DSP), a graphics processing unit (GPU), or dedicated logic circuitry such as an ASIC or FPGA.

The term “user interface” refers to any hardware and software components that enable a user to input information to, and receive information from, the system, including but not limited to touch screens, keyboards, mice, microphones, speakers, display screens, and graphical user interface (GUI) elements.

The term “cause” refers to information indicating a presumed or known reason for a medical condition or symptom, such as an illness, an injury, a vaccination, an exposure event, or any other triggering factor.

The term “symptoms” refers to manifestations or signs of a physical or mental condition experienced or observed by a user or a patient, such as fever, vomiting, diarrhea, pain, loss of consciousness, shortness of breath, or similar clinical features.

The term “elapsed time” refers to a duration between an onset of symptoms or a triggering event and a time at which information is input to the system, represented in any suitable unit such as minutes, hours, or days.

The term “generative artificial intelligence model” refers to a machine learning or artificial intelligence model configured to generate outputs, including but not limited to text, based on input data, such as a large language model or other generative model trained on large datasets.

The term “prompt sentence” refers to text data or a structured text input that specifies instructions, context, or questions provided to the generative artificial intelligence model to cause the generative artificial intelligence model to perform an analysis or generate an output.

The term “analysis result” refers to information generated by the generative artificial intelligence model in response to a prompt sentence, including but not limited to inferred medical conditions, risk assessments, or other evaluative information related to the input data.

The term “degree of urgency for visiting a medical institution” refers to an indication, which may be numerical, categorical, or qualitative, representing a level of priority or immediacy with which a user or patient should seek medical evaluation or treatment at a clinic, hospital, or similar facility.

The term “feedback” refers to information provided by a user after receiving a triage result or recommendation from the system, including but not limited to evaluations of appropriateness, actual outcomes, supplementary comments, or corrections.

The term “emergency responder” refers to a person, organization, or service that provides urgent medical or rescue assistance, such as an ambulance service, an emergency call center, or an emergency department staff member.

The term “contact content” refers to information generated for communication to an emergency responder, including but not limited to a summary of the user's or patient's condition, symptoms, estimated urgency, and recommended actions.

### BRIEF DESCRIPTION OF THE DRAWINGS

Exemplary embodiments of the present disclosure will be described in detail based on the following figures, wherein:

FIG. 1 is a schematic diagram illustrating an example of a configuration of a data processing system according to a first exemplary embodiment;

FIG. 2 is a schematic diagram illustrating an example of relevant functions of a data processing device and a smart device according to the first exemplary embodiment;

FIG. 3 is a schematic diagram illustrating an example of a configuration of a data processing system according to a second exemplary embodiment;

FIG. 4 is a schematic diagram illustrating an example of relevant functions of a data processing device and smart glasses according to the second exemplary embodiment;

FIG. 5 is a schematic diagram illustrating an example of a configuration of a data processing system according to a third exemplary embodiment;

FIG. 6 is a schematic diagram illustrating an example of relevant functions of a data processing device and a headset-type terminal according to the third exemplary embodiment;

FIG. 7 is a schematic diagram illustrating an example of a configuration of a data processing system according to a fourth exemplary embodiment;

FIG. 8 is a schematic diagram illustrating an example of relevant functions of a data processing device and a robot according to the fourth exemplary embodiment;

FIG. 9 illustrates an emotion map mapping plural emotions;

FIG. 10 illustrates an emotion map mapping plural emotions;

FIG. 11 is a sequence diagram showing the flow of data processing system processing in Example 1;

FIG. 12 is a sequence diagram showing the flow of data processing system processing in Application Example 1;

FIG. 13 is a sequence diagram showing the flow of data processing system processing in Example 2; and

FIG. 14 is a sequence diagram showing the flow of data processing system processing in Application Example 2.

### DETAILED DESCRIPTION

Description follows regarding an example of exemplary embodiments of a system according to technology disclosed herein, with reference to the appended drawings.

First, explanation follows regarding terminology employed in the following description.

In the following exemplary embodiments, a reference-numeral-appended processor (hereinafter simply referred to as “processor”) may be implemented by a single computation unit, and may be implemented by a combination of plural computation units. The processor may be implemented by a single type of computation unit, or may be implemented by a combination of plural types of computation units. Examples of computation unit include a central processing unit (CPU), a graphics processing unit (GPU), a general-purpose computing on graphics processing units (GPGPU), an accelerated processing unit (APU), and the like.

In the following exemplary embodiments, random access memory (RAM) appended with a reference numeral is memory temporarily stored with information, and is employed as working memory by a processor.

In the following exemplary embodiments, reference-numeral-appended storage is a single or plural non-volatile storage devices for storing various programs and various parameters and the like. Examples of non-volatile storage devices include flash memory (such as a solid state drive (SSD)), a magnetic disk (for example, a hard disk), magnetic tape, and the like.

In the following exemplary embodiments, a reference-numeral-appended communication interface (I/F) is an interface including a communication processor and an antenna or the like. The communication I/F has the role of communicating between plural computers. An example of a communication standard applied for the communication I/F is a wireless communication standard, such as a Fifth Generation Mobile Communication System (5G), Wi-Fi (registered trademark), Bluetooth (registered trademark), and the like.

In the following exemplary embodiments “A and/or B” has the same definition as “at least one out of A or B”. Namely, “A and/or B” may mean A alone, may mean B alone, or may mean a combination of A and B. Moreover, similar logic to “A and/or B” is applied when “and/or” is employed to link three or more items in the present specification.

### First Exemplary Embodiment

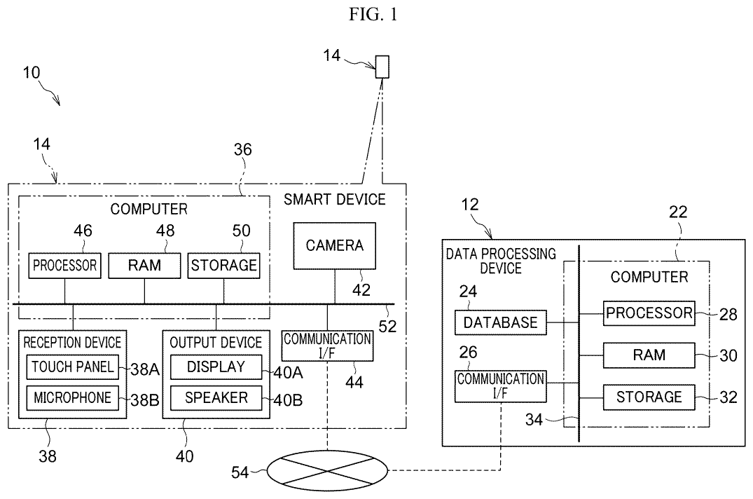

FIG. 1 illustrates an example of a configuration of a data processing system 10 according to a first exemplary embodiment.

As illustrated in FIG. 1, the data processing system 10 includes a data processing device 12 and a smart device 14. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart device 14 includes a computer 36, a reception device 38, an output device 40, a camera 42, and a communication I/F 44. The computer 36 includes a processor 46, RAM 48, and storage 50. The processor 46, the RAM 48, and the storage 50 are connected to a bus 52. The reception device 38, the output device 40, the camera 42, and the communication I/F 44 are also connected to the bus 52.

The reception device 38 includes a touch panel 38A, a microphone 38B, and the like for receiving user input. The touch panel 38A receives user input from contact of a pointer (for example, a pen, a finger, or the like) by detecting contact of the pointer. The microphone 38B receives spoken user input by detecting speech of the user. A control unit 46A in the processor 46 transmits data representing the user input received by the touch panel 38A and the microphone 38B to the data processing device 12. A specific processing unit 290 in the data processing device 12 acquires the data indicating the user input.

The output device 40 includes a display 40A, a speaker 40B, and the like for presenting data to a user 20 by outputting the data in an expression format perceivable by the user 20 (for example, audio and/or text). The display 40A displays visual information such as text, images, or the like under instruction from the processor 46. The speaker 40B outputs audio under instruction from the processor 46. The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like.

The communication I/F 44 is connected to the network 54. The communication I/F 44 and the communication I/F 26 perform the role of exchanging various information between the processor 46 and the processor 28 over the network 54.

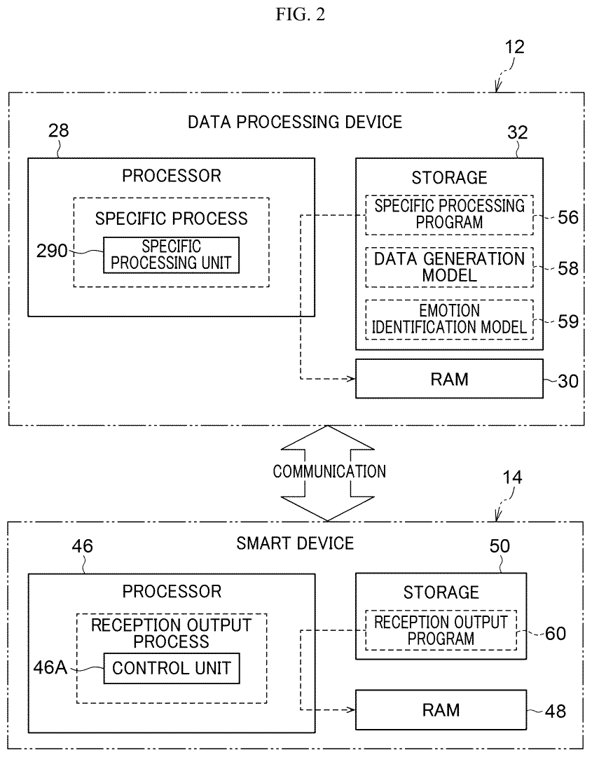

FIG. 2 illustrates an example of relevant functions of the data processing device 12 and the smart device 14.

As illustrated in FIG. 2, specific processing is performed by the processor 28 in the data processing device 12. A specific processing program 56 is stored in the storage 32. The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

A data generation model 58 and an emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart device 14. A reception and output program 60 is stored in the storage 50. The reception and output program 60 is employed by the data processing system 10 in combination with the specific processing program 56. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48. Note that a configuration may be adopted in which a similar data generation model and emotion identification model to the data generation model 58 and the emotion identification model 59 are included in the smart device 14, and these models are used to perform similar processing to the specific processing unit 290. The reception and output program is implemented by the processor 46 operating as the control unit 46A according to the reception and output program 60 executed in the RAM 48.

Note that devices other than the data processing device 12 may include the data generation model 58. For example, a server device (for example, a generation server) may include the data generation model 58. In such cases, the data processing device 12 performs communication with the server device including the data generation model 58 to obtain a processing result (prediction result or the like) obtained using the data generation model 58. The data processing device 12 may be a server device, and may be a terminal device owned by the user (for example, a mobile phone, a robot, a home electrical appliance, or the like). Next, description follows regarding an example of processing by the data processing system 10 according to the first exemplary embodiment.

### Example 1

Description follows regarding a flow of the specific processing in an Example 1. The units of the system described below are implemented by the data processing device 12 and the smart device 14. The data processing device 12 is called a “server” and the smart device 14 is called a “terminal”.

Conventional computer-implemented triage systems that assist a user in deciding whether and when to visit a medical facility typically rely on static rule sets, manually designed decision trees, or simple scoring functions. Such systems are limited in their ability to flexibly interpret free-form symptom descriptions, to adapt to complex patterns in heterogeneous clinical data, and to generate explanations that are understandable to a layperson. As a result, conventional systems often exhibit poor usability, reduced prediction accuracy, and lack of transparency, which can lead to inappropriate medical visits, unnecessary burden on medical institutions, and user distrust in automated decision aids.

Further, existing computer systems that incorporate machine learning models for medical risk estimation generally separate numerical prediction from explanation. In such systems, the prediction logic produces a risk score or class label, while any explanatory text is either predefined or minimally templated. This architectural separation leads to low integration between the numerical inference pipeline and the natural-language generation pipeline, causing latency, inconsistent outputs, and high engineering overhead. In addition, these systems rarely leverage feedback from users (such as whether a recommendation was followed or whether it was perceived as useful) in a closed-loop fashion to improve the underlying models and weighting parameters over time.

Moreover, even when generative models are used, conventional systems typically issue static, hand-crafted prompts that do not fully exploit structured outputs of upstream machine learning models, such as disease-type estimates, injury-severity levels, and time-related features. This results in generic or misaligned explanations that are not well calibrated to the user's current state, and that cannot consistently reflect computed urgency indices. Likewise, when high-risk conditions are detected, emergency escalation is often managed by separate subsystems without programmatic synthesis of the contact content from both structured and unstructured user data, leading to fragmented workflows and increased risk of information loss.

Accordingly, there is a need for improved computer technology that (i) tightly integrates structured data processing, machine learning-based estimation, and generative modeling in a unified pipeline; (ii) dynamically constructs prompt sentences for a generative model based on numeric inference outputs such as disease-type information, injury-severity information, and visit-urgency indices; (iii) provides a consistent, low-latency architecture for generating natural-language explanations and recommended actions directly aligned with underlying model outputs; and (iv) uses feedback information, including user evaluations and actual action histories, to refine machine learning parameters and weighting parameters, thereby improving the precision, robustness, and efficiency of the system over time. The present invention addresses these problems as a computer-technology improvement in how servers process state information, orchestrate heterogeneous models, and generate and present medically oriented decision support to end users.

The specific processing by the specific processing unit 290 of the data processing device 12 in Example 1 is realized by the following means.

The present invention provides a server comprising a processor, a storage device, and a communication device, the processor being configured to cause a user terminal to present, on a display device, a user interface for receiving a plurality of types of state information including cause information, symptom information, and elapsed-time information related to a health condition; to receive, via the communication device, the state information transmitted from the user terminal, convert the state information into structured data, store the structured data in the storage device, and preprocess the structured data to generate numerical feature data; to input the numerical feature data into a machine learning model stored in the storage device to estimate disease-type information and injury-severity information, and calculate a visit-urgency index on the basis of the disease-type information, the injury-severity information, the elapsed-time information, and predetermined weighting parameters stored in the storage device; to generate, on the basis of the disease-type information, the injury-severity information, and the visit-urgency index, a prompt sentence that instructs a generative AI model to perform analysis and natural-language explanation generation, to input the prompt sentence into the generative AI model, and obtain analysis-result information including explanation information in natural language understandable to the user and recommended medical-action information; and to cause the user terminal to visually present, in the user interface, at least the visit-urgency index and the analysis-result information so as to support an action selection by the user. This enables an integrated computer-implemented pipeline that transforms heterogeneous symptom inputs into structured numerical features, performs model-based medical risk estimation, and dynamically orchestrates a generative model through tailored prompt sentences, thereby improving the technical performance of the server in terms of inference accuracy, responsiveness, and consistency of generated explanations, and providing a more effective and adaptive medical triage support system.

The term “user terminal” refers to an information processing device including at least a display device, an input device, and a communication function, operated by a user to input state information and to receive and display analysis results from a server.

The term “display device” refers to a hardware component configured to visually present a user interface, analysis results, and recommendations, and includes, for example, a screen of a portable device, a monitor of a stationary device, or any equivalent visual output apparatus.

The term “user interface” refers to a logical and graphical arrangement of input and output elements presented on the display device, through which a user inputs state information and receives the visit-urgency index, analysis-result information, and recommendations.

The term “state information” refers to information related to a health condition of a user, and includes, at least, cause information, symptom information, and elapsed-time information, and may further include additional contextual, demographic, or environmental information.

The term “cause information” refers to information representing an event or factor that is associated with the onset of a symptom or health condition, such as an accident, physical impact, exposure, or similar origin.

The term “symptom information” refers to information representing perceived physical or mental abnormalities experienced by the user, including, for example, pain, swelling, dizziness, or other subjective or observable manifestations.

The term “elapsed-time information” refers to information representing a duration from an onset of a symptom or a health condition to a time at which the state information is input, including, for example, hours, minutes, or other temporal measures.

The term “communication device” refers to a hardware and software subsystem that enables data exchange between the server and the user terminal, and includes, for example, network interfaces, communication protocols, and transmission control functions.

The term “structured data” refers to state information that has been transformed into a predefined data format with explicitly defined fields and data types, suitable for storage in a storage device and for numerical processing by a machine learning model.

The term “storage device” refers to a hardware component or combination of components configured to store structured data, models, parameters, and configuration information, and includes, for example, non-volatile storage and volatile storage.

The term “numerical feature data” refers to data obtained by preprocessing the structured data so that health-related information is encoded as numerical values or vectors suitable as input to a machine learning model.

The term “preprocess” refers to a sequence of computational steps applied to structured data in order to clean, normalize, transform, or encode the data into numerical feature data.

The term “machine learning model” refers to a computational model that has been trained on training data to learn a mapping from input numerical feature data to output values, such as disease-type information and injury-severity information.

The term “disease-type information” refers to information indicating one or more candidate categories of a disease or medical condition estimated by the machine learning model from the numerical feature data.

The term “injury-severity information” refers to information indicating a severity level of a symptom or condition, including, for example, a categorical level or a numerical score, estimated by the machine learning model.

The term “visit-urgency index” refers to a numerical indicator, typically expressed as a percentage or normalized score, representing a recommended urgency with which the user should visit a medical facility.

The term “weighting parameters” refers to numerical parameters stored in the storage device and used to combine or scale intermediate quantities such as disease-type information, injury-severity information, and elapsed-time information when calculating the visit-urgency index.

The term “generative AI model” refers to an information processing model configured to generate natural-language text or other content in response to an input, and includes, for example, a generative language model that receives a prompt sentence and outputs explanation information and recommended actions.

The term “prompt sentence” refers to a sequence of characters or tokens that encodes instructions, context, and input data for the generative AI model, and that causes the generative AI model to perform analysis and generate natural-language output.

The term “analysis-result information” refers to information output by the generative AI model in response to a prompt sentence, and includes at least explanation information and recommended medical-action information.

The term “explanation information” refers to natural-language text that explains to the user, in an understandable manner, the estimated disease-type information, injury-severity information, risks, and reasoning underlying the recommended actions.

The term “recommended medical-action information” refers to natural-language text or structured data that indicates suggested actions related to seeking or not seeking medical care, such as visiting a hospital, consulting a clinic, or monitoring symptoms.

The term “action selection” refers to a decision process performed by the user, based on the visit-urgency index and the analysis-result information, to choose a particular course of behavior, such as visiting a medical facility or continuing self-observation.

The term “feedback information” refers to information obtained from the user that reflects a reaction to the analysis-result information or the recommended medical-action information, including, for example, evaluation information and actual action-history information.

The term “evaluation information” refers to information indicating the user's assessment of the usefulness, accuracy, or appropriateness of the analysis-result information or the recommended medical-action information.

The term “action-history information” refers to information representing actual actions taken by the user following receipt of the analysis-result information or recommended medical-action information, such as whether the user visited a facility and when.

The term “state-information history” refers to a collection of past state information, analysis-result information, feedback information, and related records associated with one or more users and stored in the storage device.

The term “emergency-contact text” refers to text data intended to be sent to an emergency responder or emergency-response device, including summary information of a user's condition, visit-urgency index, and other relevant contact content.

The term “emergency responder” refers to an entity capable of providing urgent or immediate assistance in a health-related emergency, including, for example, medical professionals, emergency services, or institutional response centers.

The term “emergency-response device” refers to a device or system configured to receive the emergency-contact text and initiate or coordinate an emergency response, including, for example, communication servers, dispatch systems, or equivalent apparatus.

In one embodiment, a server implements the claimed system as a network-accessible medical triage support platform. The server includes at least one processor, a main memory, a non-volatile storage device, and a network interface. The server executes an operating system, such as a general-purpose server operating system, and application software implemented, for example, using a server-side application framework, a scripting language, and a database management system. The server communicates with at least one terminal operated by a user through a communication network.

A terminal operates as a user terminal and includes a display device, an input device, and a communication module. The terminal executes a web browser or an application runtime environment, and presents a user interface generated by markup languages, style languages, and client-side scripting. The user operates the input device of the terminal to enter state information related to a health condition, including cause information, symptom information, and elapsed-time information. The terminal transmits the state information to the server in a structured request format, such as a message with a body encoded in a structured data representation.

The server receives the state information via the network interface and parses the received request by executing application logic on the processor. The server converts the raw state information into structured data. For example, the server represents the state information as records in a relational database, where each record includes fields for the cause information, the symptom information, the elapsed-time information, user identifiers, and timestamps. The server stores this structured data in a storage device. The server then preprocesses the structured data to generate numerical feature data. In one example, the server uses a data analysis library to transform textual fields into numerical vectors by applying operations such as lowercasing, removal of punctuation, tokenization, mapping tokens to indices in a vocabulary, and computing term-frequency or embedding vectors. The server converts elapsed-time information into a standardized numeric quantity, such as minutes or hours, and normalizes this quantity by using preconfigured scaling parameters. The server aggregates all processed values into a fixed-length numerical feature vector, implemented as an array of floating-point numbers.

The server inputs the numerical feature data into a machine learning model stored in the storage device. In one embodiment, the machine learning model is a deep neural network having an input layer corresponding to the dimensionality of the numerical feature vector, one or more hidden layers, and an output layer providing probability distributions over disease-type categories and severity levels. The server may implement the neural network using a numerical computation framework. The hidden layers can include fully connected layers with non-linear activation functions such as rectified linear units, and optionally normalization layers and dropout layers to improve generalization. The output layer can include a softmax activation for disease-type classification and another output head for injury-severity estimation, such as a softmax for discrete severity levels or a linear unit for continuous severity scores.

The server loads the trained model parameters, including weights and biases of each layer, from the storage device into main memory when starting the application. During inference, the server performs forward propagation by executing matrix multiplications and activation functions on the numerical feature data. The server obtains, as disease-type information, a set of class probabilities and a predicted disease-type label, and, as injury-severity information, a severity score or severity category.

The server calculates a visit-urgency index based on the disease-type information, the injury-severity information, the elapsed-time feature, and stored weighting parameters. For example, the server maintains a weighting parameter vector in the storage device, in which each weight corresponds to a clinical or technical factor such as severity level, probability of serious disease, or duration of symptoms. The server computes a weighted sum of factors, applies a normalization function, and outputs a scalar urgency value between 0 and 1 or between 0 and 100. The server may also apply piecewise scaling or non-linear mappings to reflect domain-specific priorities. By performing this computation inside the server, the system obtains a consistent and reproducible urgency index that is directly linked to the outputs of the machine learning model.

The server generates a prompt sentence to be supplied to a generative AI model. The server constructs the prompt sentence by combining textual representations of the cause information, symptom information, elapsed-time information, disease-type information, injury-severity information, and visit-urgency index according to a template or a rule-based prompt generation module. For example, the server may generate a prompt sentence such as: “The user reports swelling and pain after a fall, lasting 2 hours. The predictive model estimates a high likelihood of fracture and an 80% urgency to visit a medical facility. Explain the possible conditions, the main risks, and what the user should do next in language understandable to a non-expert.”

The server then transmits the prompt sentence to a generative AI model. In one embodiment, the generative AI model is a large-scale language model implemented as a multi-layer transformer neural network. The generative AI model includes an embedding layer that maps input tokens of the prompt sentence to vectors, multiple self-attention layers that compute contextualized representations, and an output layer that predicts a probability distribution over possible next tokens. The generative AI model parameters are pre-trained on large text corpora and optionally fine-tuned on domain-specific data related to medical explanations and triage.

The server receives, from the generative AI model, analysis-result information comprising explanation information and recommended medical-action information. The explanation information is natural-language text that describes, in user-understandable terms, possible diagnoses, key risk factors, and rationale for urgency. The recommended medical-action information is text describing suggested next steps, such as visiting a hospital immediately, visiting a clinic within a specified time, or continuing to monitor symptoms under specified conditions. The server may apply a post-processing step, such as truncation, filtering of disallowed phrases, or conversion of units, to the returned text.

The terminal receives the analysis-result information and the visit-urgency index from the server and displays them in the user interface. The terminal may visually emphasize high urgency using color or graphical indicators. The user views the displayed information and selects an action, such as searching for nearby medical facilities, initiating a call, or requesting additional details. The terminal transmits user feedback to the server, including evaluation information or action-history information.

The server uses the feedback information to improve the technical performance of the models and the urgency computation pipeline. In one embodiment, the server stores feedback information together with the state information and analysis-result information as part of a state-information history. The server periodically executes an offline or background training process in which the machine learning model parameters and the weighting parameters are updated. The server computes a loss function that compares predicted urgency indices, disease-type labels, or severity scores with ground-truth labels inferred from the user's actions and later clinical outcomes if available. The server uses gradient-based optimization, such as stochastic gradient descent or a variant with adaptive learning rates, to update model weights. By incorporating action-history information, the server calibrates the model so that future predictions align more closely with real-world outcomes, which improves prediction accuracy and reduces systematic errors.

The server can also adapt the prompt generation rules based on feedback. For instance, the server may log cases in which users report confusion or low satisfaction with explanations, and analyze these cases to refine the prompt structure, such as adding more explicit constraints (“Use short sentences.”, “Avoid technical jargon.”) or including additional context such as age group or risk factors. This adaptation improves text clarity and reduces communication errors. Because the prompt sentence is dynamically generated based on structured outputs from the machine learning pipeline, the generative AI model receives richer and more precise context than in conventional systems using static prompts, thereby improving the technical quality of generated text.

In another embodiment, the server implements an emergency escalation mechanism. When the visit-urgency index exceeds a predetermined threshold and the injury-severity information satisfies a high-risk condition, the server generates a prompt sentence that instructs the generative AI model to draft an emergency-contact text. For example, the server can generate a prompt sentence such as: “Generate a concise emergency message to a medical emergency responder. The user experienced a fall with swelling and severe pain for 2 hours. The predictive model indicates a high likelihood of fracture and an 80% urgency. Summarize the condition and recommend immediate response actions using clear, professional language.”

The generative AI model returns an emergency-contact text, which the server transmits automatically to an emergency-response device via the communication device, for instance by calling an API of a messaging system or a dispatch system. The server may also log the emergency-contact text, the time of transmission, and any follow-up responses. This integration reduces latency and human error in preparing and sending emergency communications, while maintaining consistency with the numerical risk assessment.

From a technical standpoint, the system improves computer technology in multiple ways. The server defines specific data structures for state information, numerical feature vectors, disease-type outputs, severity scores, and urgency indices, enabling efficient memory layout and batched processing in the machine learning model. The preprocessing pipeline uses vectorized operations and optimized numerical libraries to reduce computation time compared to ad hoc script-based text processing. The machine learning model architecture is designed to produce multiple outputs in a single forward pass (disease-type information and injury-severity information), thereby reducing inference latency and computation load. The weighting-based urgency calculation occurs in numerical space with pre-optimized parameters, allowing fine-grained calibration without retraining the entire neural network.

Furthermore, the tight integration between the machine learning model and the generative AI model via structured prompt sentences results in a more efficient and technically controlled generation process. The server encodes model outputs and urgency indices into the prompt sentence, which constrains the generative AI model to produce explanations logically consistent with the computed risk. This design avoids redundant recomputation of risk inside the generative model and prevents discrepancies between numerical outputs and generated text. As a result, the overall system achieves improved consistency, reduced communication overhead between subsystems, and lower processing redundancy.

The feedback-driven adaptation mechanism also constitutes a technical improvement. By treating user feedback and action-history information as additional training signals, the server adjusts internal parameters and data-flow logic to reduce errors over time. This is not merely automation of human triage but a closed-loop optimization of a computational pipeline, enabling reduced false positives and false negatives, improved calibration of urgency scores, and more appropriate allocation of computational resources to high-risk cases. For example, the server may dynamically allocate more detailed analysis and longer explanations for borderline or high-risk inputs, while using lighter-weight processing paths for low-risk inputs, thereby optimizing throughput and communication bandwidth.

In another embodiment, the server deploys the machine learning model on hardware accelerators, such as graphics processing units, and uses batch processing of multiple user requests to further improve computational efficiency. The server caches intermediate representations, such as tokenized forms of common symptoms or typical prompt sentence segments, to reduce repeated preprocessing overhead. The server can also compress and index historical records in the storage device, which enhances retrieval performance for training and analysis tasks.

Alternative configurations are possible. The machine learning model may be implemented as a gradient-boosted decision tree ensemble, a probabilistic graphical model, or a hybrid architecture combining neural networks for text encoding with tree-based models for final classification. The generative AI model may reside on a remote inference service or on a dedicated local server, and communication between the server and the generative AI model may be secured and optimized through streaming protocols. The terminal may be a portable device, a stationary workstation, or a specialized medical terminal. In each variation, the server maintains the core functions of transforming raw state information into numerical feature data, computing disease-type and severity outputs, deriving a visit-urgency index via specific weighting parameters, dynamically generating prompt sentences aligned with those outputs, and orchestrating a generative AI model to produce analysis-result information that is presented on the terminal.

Through these configurations, the server, the terminal, and the user cooperate to achieve a technical effect that goes beyond manual triage or simple rule-based scoring. The system exploits structured data encoding, model-based estimation, and controlled generative processing in a way that improves processing speed, prediction accuracy, consistency of explanations, and management of critical communications, thereby constituting an improvement to computer technology and not merely an automation of human reasoning.

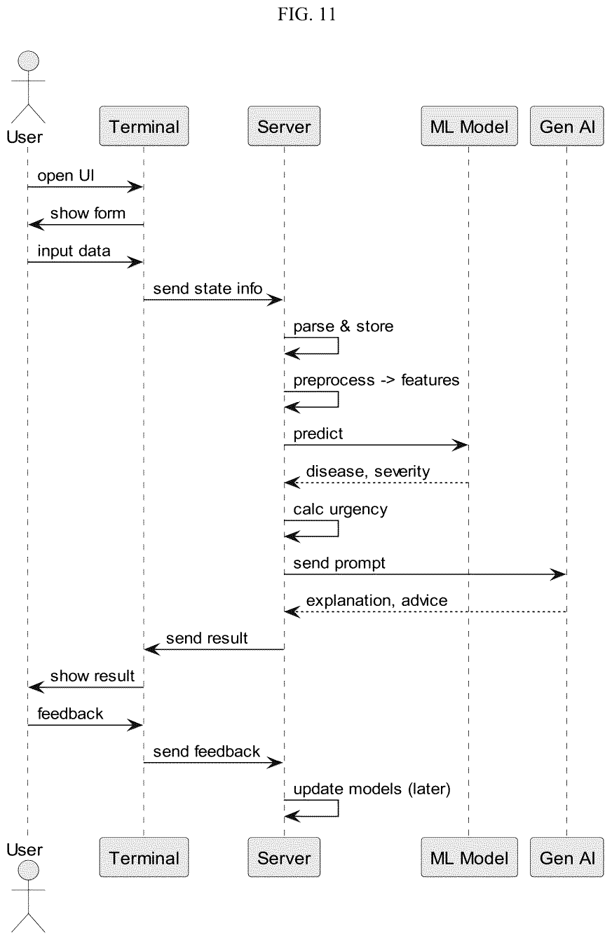

The following describes the processing flow using FIG. 11.

### Step 1

The terminal displays a user interface for health information input.

The terminal loads markup, style, and script resources from the server and renders input fields for cause information, symptom information, and elapsed-time information on the display device.

Input: No user medical data; only the interface layout definition received from the server.

Processing: The terminal executes client-side script to build the document object model, attach event listeners to input elements, and initialize an empty internal data structure (for example, a key-value map) to hold user input.

Output: A visible input form on the display that is ready to receive state information from the user.

### Step 2

The user enters state information into the user interface.

The user operates the input device of the terminal to type or select values for cause information (for example, “fall”), symptom information (for example, “swelling and pain”), and elapsed-time information (for example, “2 hours”), and then activates a submission control.

Input: Blank or default values in the input form displayed on the terminal.

Processing: The user modifies the values in the form elements by typing or selecting items, thereby updating the internal state of the fields maintained by the terminal's script environment.

Output: Completed state information present in the input form, ready for capture by the terminal.

### Step 3

The terminal validates and structures the state information.

The terminal reads the values from the input fields when the user submits the form and checks for missing or invalid entries (for example, empty symptom information or non-sensical elapsed-time information).

Input: Raw text values entered by the user in the form fields.

Processing: The terminal performs syntactic validation (such as checking non-emptiness and acceptable length), optionally normalizes formats (for example, trimming whitespace), and constructs a structured representation (for example, a dictionary with keys “cause”, “symptom”, and “elapsed_time”).

Output: A validated and structured state information object, or an error indication shown to the user if validation fails.

### Step 4

The terminal transmits the structured state information to the server.

The terminal encodes the structured state information into a request body, adds appropriate headers, and sends the request over a communication network to an application endpoint implemented on the server.

Input: The structured state information object generated in Step 3.

Processing: The terminal serializes the structured object into a transmission format, attaches routing and content metadata, and invokes the communication module to send the data using a network protocol.

Output: A network message containing the state information delivered to the server's network interface.

### Step 5

The server receives and parses the incoming state information.

The server accepts the network message via the communication device, passes it to an application handler, and extracts the state information payload.

Input: A network message that encapsulates the user's state information in a serialized format.

Processing: The server validates the message headers, deserializes the body, checks for required fields, and converts the deserialized data into internal structured data, such as a record object with typed fields for cause information, symptom information, and elapsed-time information.

Output: A structured state information record in the server's application memory, or an error response sent back to the terminal if parsing fails.

### Step 6

The server stores the state information in a storage device.

The server uses a database access layer to map the structured state information record to a persistent data record and writes it to a database.

Input: The structured state information record residing in the server's memory.

Processing: The server creates a new database entity with fields for the cause information, symptom information, elapsed-time information, a user identifier if available, and a timestamp. The server executes a write operation through a database management system and commits the transaction.

Output: A persistent record stored in the database, identified by a record identifier that can be used for subsequent retrieval.

### Step 7

The server retrieves the stored state information for analysis.

The server queries the database to obtain the newly stored record and loads it into an in-memory data structure suitable for preprocessing.

Input: A record identifier or query parameters that specify the stored state information.

Processing: The server issues a retrieval command to the database, obtains the record, and constructs a data structure, such as a row in a tabular data container, with fields corresponding to the stored attributes.

Output: A retrieved state information instance prepared for preprocessing in the server's main memory.

### Step 8

The server preprocesses the state information to generate numerical feature data.

The server transforms textual and temporal fields into numerical representations compatible with a machine learning model.

Input: The retrieved state information instance including cause information, symptom information, and elapsed-time information.

Processing: The server converts text to lowercase, removes punctuation, splits text into tokens, maps tokens to indices via a predefined vocabulary, and computes numerical vectors (for example, term-frequency or embedding vectors). The server converts elapsed-time information into a standardized numeric duration, scales it using pre-defined normalization parameters, and concatenates all numerical components into a fixed-length feature vector implemented as an array of real-valued elements.

Output: Numerical feature data corresponding to the user's health state, ready to be input to a machine learning model.

### Step 9

The server performs inference using a machine learning model.

The server inputs the numerical feature data into a trained model to estimate disease-type information and injury-severity information.

Input: The numerical feature vector produced in Step 8.

Processing: The server loads model parameters from the storage device into memory if not already loaded, organizes the feature vector into the expected tensor shape, and executes forward propagation through the model layers, including matrix multiplications and non-linear activations. The server obtains output values such as probability distributions over disease categories and severity levels. The server then selects one or more disease-type labels with highest probability and derives severity information either as a discrete class or as a continuous score.

Output: Disease-type information and injury-severity information associated with the input state information.

### Step 10

The server calculates a visit-urgency index based on model outputs and configured parameters.

The server combines the disease-type information, the injury-severity information, and the elapsed-time feature using stored weighting parameters to derive a scalar urgency value.

Input: Disease-type information, injury-severity information, the normalized elapsed-time value, and weighting parameters stored in the server's storage device.

Processing: The server maps categorical outputs to numeric severity and risk values, multiplies each value by corresponding weights, sums the weighted values, and applies a normalization or clipping function to constrain the result within a predetermined range, such as 0 to 100. The server may also apply thresholding or non-linear scaling functions to emphasize critical ranges.

Output: A visit-urgency index representing the recommended urgency to visit a medical facility.

### Step 11

The server generates a prompt sentence for a generative AI model.

The server constructs a natural-language instruction that encodes the user's state information, the model's estimates, and the calculated visit-urgency index to guide a generative AI model.

Input: Cause information, symptom information, elapsed-time information, disease-type information, injury-severity information, and the visit-urgency index.

Processing: The server applies a prompt generation template or rule set that arranges these data elements into a coherent textual instruction. For example, the server creates a prompt sentence such as: “The user reports swelling and pain after a fall, lasting 2 hours. The predictive model estimates a high likelihood of fracture and an 80% urgency to visit a medical facility. Explain the possible conditions, the main risks, and what the user should do next in language understandable to a non-expert.” The server ensures that numeric values and labels are correctly formatted and embedded into the sentence.

Output: A prompt sentence ready to be submitted to a generative AI model.

### Step 12

The server invokes the generative AI model with the prompt sentence.

The server sends the prompt sentence to a generative AI model and requests generation of explanation information and recommended medical-action information.

Input: The prompt sentence generated in Step 11.

Processing: The server transmits the prompt sentence to the generative AI model via a communication interface or local procedure call. The generative AI model processes the prompt sentence by converting tokens to embeddings, applying attention-based transformations, and generating a sequence of output tokens representing natural-language text. The server receives the generated text segments, assembles them into complete sentences or paragraphs, and optionally enforces length or content constraints.

Output: Analysis-result information that includes explanation information and recommended medical-action information expressed as natural-language text.

### Step 13

The server prepares a response for the terminal.

The server combines the visit-urgency index, the disease-type information, the injury-severity information, and the generated analysis-result information into a unified response structure.

Input: The visit-urgency index from Step 10, the disease-type information and injury-severity information from Step 9, and the analysis-result information from Step 12.

Processing: The server organizes these elements into an output data structure, associates labels or keys with each element (such as “urgency”, “diagnosis”, “severity”, “explanation”, and “recommendation”), and encodes the structure into a transmission-ready representation. The server may also include additional metadata such as timestamps or confidence scores.

Output: A response data object representing the complete analysis result, ready to be transmitted to the terminal.

### Step 14

The server transmits the analysis result to the terminal.

The server sends the response data object back to the terminal through the communication device as a reply to the original request.

Input: The response data object created in Step 13.

Processing: The server serializes the response data, attaches appropriate status indicators, and uses the network interface to deliver the message to the terminal's network endpoint.

Output: A network response that conveys the urgency index and analysis-result information to the terminal.

### Step 15

The terminal displays the analysis-result information to the user.

The terminal receives the response from the server, extracts the included elements, and renders them in the user interface.

Input: The network response containing the visit-urgency index, disease-type information, injury-severity information, and analysis-result information.

Processing: The terminal deserializes the response, updates elements of the document object model to show textual content such as “There is a high possibility of fracture” and “Visit urgency: 80%,” and displays the explanation and recommended actions produced by the generative AI model. The terminal may adjust visual styling based on urgency, such as highlighting high urgency in a distinct color.

Output: A fully updated interface presenting the computed urgency index and natural-language explanations, visible to the user.

### Step 16

The user reviews the result and selects a subsequent action.

The user reads the displayed disease-type information, urgency index, and explanation, and decides how to respond.

Input: The visual presentation of the analysis-result information on the terminal.

Processing: The user mentally evaluates the information and interacts with the terminal by selecting options such as “Call emergency services,” “Find nearby clinic,” or “Request further explanation,” which triggers new inputs into the system.

Output: User-generated follow-up actions or feedback commands entered into the terminal.

### Step 17

The terminal transmits user feedback and action-history information to the server.

The terminal collects the user's selections or evaluations and sends them as feedback information to the server.

Input: User choices, ratings, or comments entered after reviewing the analysis-result information.

Processing: The terminal structures the feedback (for example, as action type, timestamp, and evaluation score), encodes it into a message, and transmits it over the network to the server.

Output: A feedback message delivered to the server, containing evaluation information and/or action-history information.

### Step 18

The server records and utilizes feedback information for system improvement.

The server stores the feedback information and may later use it to adjust model parameters and weighting parameters.

Input: The feedback message received from the terminal in Step 17.

Processing: The server deserializes the feedback, associates it with the corresponding state information record, and writes it as part of a state-information history in the storage device. In a background or scheduled process, the server retrieves accumulated histories, computes error measures between past predictions and realized outcomes inferred from the feedback, and updates the machine learning model and weighting parameters using optimization algorithms.

Output: Updated model parameters and weighting parameters that refine future disease-type estimates, severity assessments, and visit-urgency indices.

### Application Example 1

Description follows regarding a flow of the specific processing in an Application Example 1. The units of the system described below are implemented by the data processing device 12 and the smart device 14. The data processing device 12 is called a “server” and the smart device 14 is called a “terminal”.

Conventional health assessment support systems that rely solely on either rule-based engines or end-to-end machine learning models suffer from several technical limitations when deployed on general-purpose computing platforms. First, such systems often directly pass raw user input (for example, free-text symptom descriptions and loosely formatted elapsed-time data) to a predictive model or to a generative AI model. This direct usage of unnormalized, inconsistently structured data leads to unstable inference behavior, degraded prediction accuracy, and increased computational overhead for downstream processing. Second, known systems typically use a single analytical engine, either a deterministic expert system or a single statistical model, which makes it difficult to reconcile numeric risk estimates with natural language explanations and recommendations in a consistent and auditable manner. As a result, a processor must perform ad hoc post-processing, resulting in inefficiencies in memory usage, processing time, and system maintainability.

Furthermore, existing approaches that invoke generative AI models to assist with medical or health-related assessments frequently construct prompt sentences in an ad hoc or static fashion, without taking into account structured outputs from auxiliary machine learning models such as disease classifiers or severity estimators. This lack of integration causes the generative AI model to operate with incomplete or noisy context, thereby increasing the likelihood of inconsistent outputs and requiring additional corrective computation by the host system. It also becomes difficult for the processor to systematically incorporate the generative AI output into a numerical urgency calculation, leading to redundant computations and complex, non-scalable program logic.

Additionally, feedback from users (for example, corrections to proposed disease names or evaluations of recommended actions) is often stored as unstructured logs and is not effectively leveraged to improve either the underlying machine learning models or the prompt-generation logic used for the generative AI model. This results in a static system that cannot adapt its computational behavior to real-world usage patterns, limiting the long-term accuracy and efficiency of the system.

There is therefore a need for a computer-implemented health assessment system that technically improves the way a processor acquires, normalizes, and structures health-related data, coordinates hybrid processing between a deterministic machine learning model and a generative AI model via dynamically generated prompt sentences, and computes a quantitative urgency index in a consistent and resource-efficient manner. Such a system should further enable the processor to use structured feedback to update internal models and prompt-generation strategies, thereby improving processing accuracy, reducing unnecessary computation, and enhancing the overall reliability of the health assessment workflow executed on the computing platform.

The specific processing by the specific processing unit 290 of the data processing device 12 in Application Example 1 is realized by the following means.

The present invention provides a server comprising a processor configured to provide a user interface on an information processing device to receive health-related information including at least a cause, a symptom, and an elapsed time; normalize, in the server, the received health-related information and extract feature data from the normalized information; execute, on the server, a machine learning model using the feature data to estimate a disease name and a severity level of a health condition; generate, on the server, a structured prompt sentence based on at least the received health-related information and the estimated disease name and severity level, the prompt sentence instructing a generative AI model to perform an analysis; transmit the generated prompt sentence from the server to an external generative AI model and receive, at the server, an analysis result including at least explanatory text and recommended response content; calculate, on the server, an urgency of visiting a medical institution as a numerical index by applying, to the estimated disease name and severity level and optionally to a confidence value of the machine learning model, predetermined weighting parameters and combining the result with the analysis result from the generative AI model; present, via the user interface, the calculated urgency, the estimated disease name, the estimated severity level, and the recommended response content; and store, in a storage medium accessible by the server, the health-related information, the estimated disease name and severity level, the analysis result, and user feedback information, and update at least one of the machine learning model and a generation rule set for the prompt sentence based on the stored data used as learning data. This enables the server to perform health-assessment computations in a technically improved manner by systematically structuring input data, coordinating deterministic machine learning inference with generative AI processing via optimized prompt sentences, generating a consistent and computationally efficient numerical urgency index, and adaptively refining both model parameters and prompt-generation logic based on accumulated feedback, thereby enhancing processing accuracy, resource utilization, and reliability of the overall computer-implemented health assessment workflow.

The term “system” refers to an arrangement including at least one information processing apparatus and associated components configured to execute the functions recited in the claims.

The term “processor” refers to one or more hardware processing units, such as a central processing unit or a computation core, configured to execute program instructions to perform the claimed operations.

The term “information processing device” refers to a general-purpose or special-purpose computing apparatus, including at least a processor, a memory, and an input/output interface, capable of executing software to process digital data.

The term “user interface” refers to a combination of hardware and software components, such as a display, input devices, and graphical or textual screens, through which a user inputs information and receives information from the system.

The term “health-related information” refers to digital data describing a physical or mental condition of a user, including at least a cause of the condition, a symptom, and an elapsed time from onset, and optionally additional contextual information.

The term “cause” refers to data indicating a factor, event, or condition that is considered to be associated with the onset of a health condition experienced by the user.

The term “symptom” refers to data describing a perceived physical or psychological change or abnormality associated with a health condition of the user.

The term “elapsed time” refers to data indicating a duration from a starting point of a symptom or health event to a current time at which the information is input.

The term “normalize” refers to processing input data to convert it into a standardized, consistent, and machine-readable format, including operations such as unit conversion, text cleaning, tokenization, or numerical scaling.

The term “feature data” refers to structured numerical or categorical values derived from raw input data and suitable for input into a machine learning model.

The term “machine learning model” refers to a computational model that has been trained on example data to learn relationships between input features and output targets, and that is configured to generate predictions such as a disease name or a severity level when executed by a processor.

The term “disease name” refers to a label indicating a type of medical condition or illness that is estimated by the system based on input features.

The term “severity level” refers to a qualitative or quantitative indicator representing an estimated seriousness or impact of a health condition.

The term “generative AI model” refers to a computational model, such as a large language model, configured to generate natural language or other structured output content in response to a textual or symbolic input.

The term “prompt sentence” refers to a structured text string that is generated by the system and supplied as input to the generative AI model to instruct the generative AI model to perform a specified analysis or content generation.

The term “analysis result” refers to output information produced by the generative AI model in response to a prompt sentence, including at least explanatory text and recommended response content.

The term “explanatory text” refers to a natural-language description generated by the generative AI model that explains a possible health condition, its background, or its implications.

The term “recommended response content” refers to natural-language guidance generated by the generative AI model that suggests specific actions or behaviors for the user in view of the estimated health condition.

The term “urgency of visiting a medical institution” refers to a numerical index, such as a percentage or score, indicating a recommended priority or immediacy for the user to seek medical care.

The term “weighting parameters” refers to numeric values associated with specific disease names, severity levels, or model confidence values, which are used by the system to compute the urgency of visiting a medical institution.

The term “confidence value” refers to a numeric measure indicating a level of certainty or probability associated with a prediction output by the machine learning model.

The term “storage medium” refers to a non-transitory computer-readable medium, such as a semiconductor memory, a magnetic disk, or an optical disk, configured to store programs and data used by the system.

The term “learning data” refers to data used to train or update a machine learning model or to adjust rules for generating a prompt sentence, including stored input information, output results, and feedback.

The term “feedback information” refers to data provided by a user that evaluates, confirms, or corrects a prediction or recommendation produced by the system.

The term “generation rule set for the prompt sentence” refers to a collection of conditions, templates, and parameter values that determine how a prompt sentence is constructed based on input data and intermediate results.

The term “communication function” refers to hardware and software components that enable transmission and reception of digital data between the system and external devices or services over a communication network.

The term “emergency destination” refers to an external contact point, such as an emergency service, a medical institution, or a responsible person, designated to receive automatically transmitted contact content in urgent situations.

The term “contact content” refers to a message including at least emergency contact information and current symptom information, generated for transmission to an emergency destination.

In one embodiment, a server executes a health assessment application on a general-purpose computing platform comprising at least one processor, a main memory, a non-transitory storage device, and a network interface. The server runs an operating system such as a generic server operating system, a web application framework such as a generic web framework, and a machine learning library such as a generic machine learning library. The server further communicates with a generative AI model hosted on an external computation environment via a network such as the Internet.

A terminal operates as a client device and includes at least a processor, a memory, a communication interface, a display device, and an input device. The terminal runs a web browser or a dedicated application that presents a user interface generated by the server. The terminal may be implemented by a smartphone, a tablet, a notebook computer, or another portable or stationary information processing device.

A user operates the terminal to input health-related information. The user interface on the terminal presents input fields for at least a cause, a symptom, and an elapsed time. The terminal transmits the entered information to the server over a secure communication protocol. The terminal also receives, from the server, a disease name, a severity level, an urgency of visiting a medical institution, and recommended response content, and displays such information to the user on the display device.

The server stores, in the non-transitory storage device, program modules including a user interface module, a preprocessing module, a feature extraction module, a machine learning inference module, a prompt generation module, a generative AI interface module, a risk calculation module, a feedback processing module, a model update module, and a logging module. The server loads these modules into memory and executes them by the processor.

The server uses the preprocessing module to normalize health-related information received from the terminal. The server converts elapsed-time strings into numeric values, such as hours from symptom onset, and standardizes textual symptom descriptions by applying lowercasing, punctuation removal, and tokenization. The server maps common synonyms or lay terms for symptoms to canonical symptom descriptors using a lookup table stored in the storage device. This normalization produces a consistent internal data structure, for example, a record with fields for canonical cause codes, canonical symptom tokens, and numeric elapsed-time values.

The server uses the feature extraction module to convert normalized health-related information into feature data suitable for machine learning processing. The server applies a text vectorization algorithm, such as term frequency-inverse document frequency, to canonical symptom tokens and cause-related tokens. The server further includes numeric features such as elapsed-time values, age bands, or binary flags indicating the presence of specific high-risk symptom patterns. The server constructs feature vectors as arrays of floating-point values and stores them temporarily in memory.

The server uses the machine learning inference module to estimate a disease name and a severity level from the feature vectors. In one embodiment, the server employs a supervised classifier trained on labeled health records. The server may use a linear model, an ensemble model, or a neural network model. For example, the server may implement a multilayer neural network including an input layer corresponding to the feature vector, one or more hidden layers with nonlinear activation functions, and an output layer representing disease classes. The server trains the neural network by minimizing a cross-entropy loss function using gradient-based optimization and updates weight parameters through backpropagation. The server stores the trained weights and model architecture in the storage device and loads them into memory at runtime.

The server also uses a separate severity estimation model or an output head within the same neural network architecture to predict a severity level such as mild, moderate, or severe. The server may model severity as ordinal categories and train the severity estimator using a loss function that reflects the ordered nature of severity levels. The server outputs predicted probability distributions for both disease classes and severity levels, and obtains a disease label and a severity label by selecting classes with maximum probability values.

The server employs the risk calculation module to calculate an urgency of visiting a medical institution. The server stores, in a configuration repository, weighting parameters that associate each disease class and each severity level with numeric risk weights. The server computes a base risk score as a weighted combination of disease and severity weights. The server optionally incorporates model confidence values by scaling the base risk score according to the predicted probabilities. The server normalizes the resulting score into a bounded numeric range, such as 0 to 100, and determines an urgency percentage. By relying on explicitly defined weighting parameters and probabilistic outputs, the server achieves a consistent and computationally efficient method for calculating urgency across different disease and severity combinations.

The server uses the prompt generation module to construct a prompt sentence for the generative AI model. The server composes the prompt sentence using a template stored in the storage device, and fills in fields with the normalized input information, the predicted disease name, the predicted severity level, and the computed urgency percentage. The server ensures that the prompt sentence explicitly instructs the generative AI model to perform specific tasks and to output results in a structured format. A representative prompt sentence used by the server is for example:
“Symptoms: headache, dizziness
Cause: unknown
Elapsed time: 2 hours
The internal machine learning models predict the following:
Disease: mild dehydration (tentative)
Severity: mild
Urgency: 30%

You are a generative AI model that assists with health assessments for employees during work. Based on this information, please do the following:
1. Briefly explain the most likely condition.
2. Confirm or refine the disease name.
3. Confirm or refine the severity level.
4. Provide clear and practical recommendations for the user's next actions at work, including whether to rest, hydrate, or visit a medical institution.

Output your answer in English with the following structure:
Disease name:
Severity:
Urgency (%):
Recommendation:”

The server uses the generative AI interface module to transmit the prompt sentence to the generative AI model via a network. In one embodiment, the generative AI model is a large language model that implements a transformer-based neural network architecture. The external environment hosting the model may contain multiple processing units and specialized acceleration hardware. The generative AI model has been pre-trained on large textual datasets and optionally fine-tuned on health-related text. The model uses self-attention layers to encode the prompt sentence and generates output tokens sequentially based on learned conditional probability distributions. The server provides the prompt sentence as input to the model and receives an output text that includes a disease name, a severity description, an urgency expression, and recommended response content.

The server uses the result interpretation logic in the generative AI interface module to parse the output text. The server identifies lines beginning with specific markers such as “Disease name:”, “Severity:”, “Urgency (%):”, and “Recommendation:”, and extracts corresponding content. The server optionally compares an urgency percentage suggested by the generative AI model with the internally computed urgency percentage, and in some embodiments the server enforces consistency by retaining the internal numeric value while adopting narrative explanations and recommendations from the generative AI output.

The server uses the user interface module to transmit, to the terminal, a response including the disease name, the severity level, the urgency percentage, and the recommendation text. The terminal displays the numeric urgency, for example as “Urgency: 30%”, together with the predicted disease name and a short explanation. The terminal further shows recommended actions such as “Please drink water and rest. If the headache or dizziness continues or worsens, visit a medical institution.”

The user reviews the displayed information, evaluates their health condition, and may follow the recommended actions. The user may also provide feedback using the terminal. The user may, for example, indicate that a suggested disease name is inaccurate, that a severity level seems too high or too low, or that a recommendation was not followed. The terminal sends this feedback information to the server.

The server uses the feedback processing module to analyze feedback information. The server associates the feedback with the original feature vectors, predicted outputs, and generative AI responses stored in the storage device. The server uses this collected dataset as learning data for subsequent model improvements. For example, the server may retrain the classification model on an extended dataset that incorporates feedback-labeled records, or may adjust weighting parameters used in the urgency calculation. The server may also modify the prompt-generation rules by altering template wording or requesting more constrained output formatting from the generative AI model when recurring failure patterns are observed.

The server uses the model update module to periodically update machine learning parameters and prompt-generation templates. In one embodiment, the server performs offline retraining by loading historical interaction logs and feedback from the storage device, shuffling and augmenting the data, and retraining neural network models using mini-batch gradient descent. The server may use regularization techniques such as dropout or weight decay to prevent overfitting. The server may introduce data augmentation techniques such as synonym replacement or paraphrasing of symptom descriptions to improve robustness to linguistic variation. The server then deploys the updated models by storing new weight parameters, updating version information, and reloading the models in memory.

The server achieves technical improvements over conventional approaches through the described architecture. By normalizing input data and converting it into structured feature vectors, the server reduces variability and ambiguity that would otherwise burden both deterministic models and generative models. By using explicit weighting parameters and model confidence scores to compute an urgency index, the server implements a transparent and computationally efficient decision mechanism, rather than relying solely on opaque natural language outputs. By dynamically generating optimized prompt sentences that incorporate structured predictions, the server reduces the token length and contextual noise presented to the generative AI model, thereby improving generation quality and reducing network and computation load. By storing structured logs and feedback and using them to update both the machine learning models and prompt-generation rules, the server incrementally enhances accuracy and processing efficiency without requiring manual rule rewriting.

The server's hybrid processing pipeline, in which a deterministic machine learning model and a generative AI model are coordinated through an explicitly defined prompt sentence, enables non-trivial improvements in computational behavior. For example, the server can pre-filter low-risk cases using the machine learning model and apply narrower templates for the generative AI model, thereby reducing the volume of generated text and accelerating overall response time. The server can also control model behavior by enforcing a fixed output format, which facilitates rapid parsing and minimizes post-processing overhead. These measures result in faster, more reliable updates to the user interface and reduced resource consumption on both the server and the external generative AI environment.

In an alternative embodiment, the server executes a different type of machine learning model, such as a probabilistic graphical model or an ensemble of trees. The server may use gradient boosting trees to estimate disease risk and severity and still use the same prompt-generation and generative AI integration strategy. In another embodiment, the server deploys the generative AI model on-premises rather than relying on an external environment. In this case, the server uses local computation units, potentially with specialized accelerators, and the generative AI interface module invokes the model through an internal application programming interface instead of a network call.

In another embodiment, the terminal executes a native application rather than a web browser. The terminal application can localize some preprocessing, such as basic normalization and validation of input data, thereby reducing the size and complexity of messages sent to the server. The server still performs the core machine learning and generative AI integration functions, and the resulting data structures remain compatible with the server-side modules described above.

In yet another embodiment, the server may incorporate additional sensor-derived data, such as heart rate measurements or activity patterns collected via wearable devices connected to the terminal. The server extends the feature vectors with these physiological and behavioral features, allowing more accurate disease and severity estimation. The server's normalization and feature extraction modules adapt to treat these sensors'readings with appropriate scaling and temporal aggregation. The weighting parameters for urgency calculation are extended to account for sensor-based risk factors, thereby further improving the technical accuracy and robustness of the urgency index.

Through these embodiments, the server, the terminal, and the user cooperate to implement a health assessment system that improves the operation of the underlying computer technology. The system reduces data inconsistency, improves machine inference stability, optimizes use of generative AI resources through targeted prompt sentences, and systematically exploits feedback for continual model optimization. These technical features lead to higher accuracy, reduced processing time, and lower communication load compared to systems that directly use raw input or rely solely on unstructured generative outputs, thus demonstrating that the invention is not limited to abstract data manipulation but provides concrete improvements to computer-based health assessment processing.

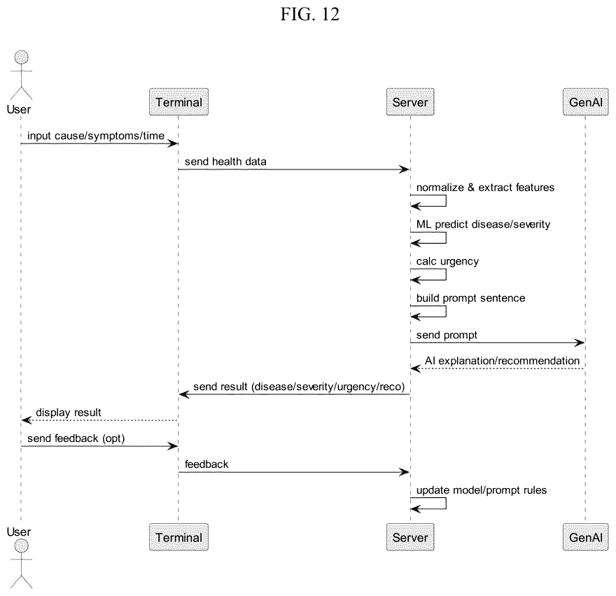

The following describes the processing flow using FIG. 12.

### Step 1

User operates the terminal to access a health assessment screen provided by the server.

User views input fields on the terminal for at least a cause, one or more symptoms, and an elapsed time from symptom onset.

Input: No prior medical data is required; the user only needs access to the terminal and the displayed user interface.

Output: The user provides raw text entries such as “unknown” for cause, “headache, dizziness” for symptoms, and “2 hours” for elapsed time into the input fields.

### Step 2

Terminal acquires the text values entered in the input fields from the user interface components.

Terminal temporarily stores the values in memory associated with a document object model of the user interface.

Terminal may perform basic validation, such as checking that mandatory fields are not empty and that elapsed time conforms to a recognizable format (for example, a number followed by “minutes” or “hours”).

Input: Raw user-entered strings for cause, symptoms, and elapsed time.

Output: A structured set of validated key-value pairs, for example {cause: “unknown”, symptoms: “headache, dizziness”, elapsed_time: “2 hours”}.

### Step 3

Terminal converts the structured key-value pairs into a machine-readable message and transmits the message to the server over a network connection.

Terminal formats the message using a structured representation such as a JSON-like object and attaches metadata such as a timestamp and a user identifier or session identifier.

Terminal sends the message via a secure communication protocol to an application programming interface endpoint exposed by the server.

Input: Validated key-value pairs representing the user's cause, symptoms, and elapsed time.

Output: A network request containing a structured payload that arrives at the server and is ready to be parsed.

### Step 4

Server receives the network request through a communication interface and forwards the payload to an application module in memory.

Server parses the structured payload to extract individual fields for cause, symptoms, elapsed time, and any auxiliary metadata.

Server stores the parsed input temporarily in a data structure residing in main memory for further processing.

Input: Structured payload transmitted from the terminal, including the cause, symptoms, and elapsed time as strings.

Output: An internal representation of the user's health-related information as program variables or records, for example an object with canonical field names for cause, symptoms, and elapsed time.

### Step 5

Server normalizes the received health-related information using a preprocessing module.

Server converts the elapsed-time string into a numeric duration, such as total minutes or hours from onset, by parsing numerical values and time units and applying appropriate multiplication factors.

Server processes the symptom and cause text by applying transformations such as lowercasing, removal of extraneous punctuation, tokenization into words or phrases, and mapping of synonyms to standard terms using a stored mapping table.

Input: Internal record containing raw strings for cause, symptoms, and elapsed time.

Output: A normalized record containing standardized symptom terms, standardized cause terms, and a numeric elapsed-time value.

### Step 6

Server generates feature data from the normalized record using a feature extraction module.

Server transforms symptom and cause tokens into numerical vectors by consulting a stored vocabulary and computing vector components such as term frequency-inverse document frequency values.

Server combines these textual feature components with numerical features, such as elapsed-time values or binary indicators for specific symptom categories, into a single feature vector represented as an ordered list of numerical values.

Input: Normalized record including standardized symptom tokens, cause tokens, and numeric elapsed time.

Output: A feature vector suitable as input to a machine learning model, for example an array of real-valued components.

### Step 7

Server applies one or more machine learning models to the feature vector to estimate a disease name and a severity level.

Server loads model parameters from storage into memory, including weight matrices and bias values for a neural network or parameters for another classifier.

Server performs a forward-pass computation where the feature vector is multiplied by weight matrices, combined with biases, and passed through nonlinear activation functions layer by layer until output values representing probabilities for disease classes and severity levels are obtained.

Server selects the disease class with the highest probability as the estimated disease name and selects a severity class with the highest probability as the estimated severity level.

Input: Feature vector generated from normalized health-related information.

Output: Estimated disease name, estimated severity level, and associated probability distributions or confidence values.

### Step 8

Server calculates an urgency of visiting a medical institution as a numerical index using a risk calculation module.

Server retrieves preconfigured risk weights associated with each disease class and each severity level from a configuration data store.

Server combines the disease-specific weight and the severity-specific weight with a function of the model's confidence values, for example by computing a weighted sum or weighted product and then normalizing the result to a predefined scale such as 0 to 100.

Server rounds the normalized score to produce an integer urgency percentage that represents a recommended urgency level for medical consultation.

Input: Estimated disease name, estimated severity level, and confidence values, together with stored risk weighting parameters.

Output: A numeric urgency index, for example an urgency percentage such as 30%.

### Step 9

Server constructs a prompt sentence for a generative AI model using a prompt generation module.

Server selects a template that defines the structure and instructions of the prompt sentence and inserts the normalized input information, the estimated disease name, the estimated severity level, and the calculated urgency index into specified positions in the template.

Server ensures that the prompt sentence contains explicit instructions to the generative AI model to provide an explanation of the condition, refine or confirm the estimates, and generate recommended response content in a prescribed output format.

Input: Normalized health-related information, estimated disease name, estimated severity level, and numeric urgency index.

Output: A composed prompt sentence as a text string ready to be transmitted to the generative AI model.

### Step 10

Server transmits the prompt sentence to an external generative AI model via a network interface using a generative AI interface module.

Server embeds the prompt sentence into a request message that conforms to a predefined application programming interface for the generative AI model, along with parameters such as maximum output length or temperature for generation stochasticity.

Server sends the request to the generative AI environment and waits for a response while monitoring for timeouts or errors.

Input: Prompt sentence describing the user's case, including cause, symptoms, elapsed time, estimated disease name, severity level, and urgency.

Output: A response message received from the generative AI model containing generated text that explains the condition, describes severity, suggests urgency, and recommends concrete actions.

### Step 11

Server interprets the generated text returned by the generative AI model.

Server identifies segments of the generated text according to markers defined in the prompt sentence, such as lines starting with “Disease name:”, “Severity:”, “Urgency (%):”, and “Recommendation:”.

Server extracts these segments as separate fields and, when necessary, converts any urgency expression from text or percentage format into a numeric representation for consistency with the internally computed urgency.

Server optionally compares the urgency suggested by the generative AI model with the previously calculated urgency index and resolves inconsistencies by, for example, adopting the internal numeric index while using the generative AI explanation primarily for narrative purposes.

Input: Generated text from the generative AI model.

Output: Parsed values for a refined disease name, refined severity description, optional urgency information, and recommendation text structured into distinct fields.

### Step 12

Server compiles a final assessment result for transmission to the terminal.

Server combines the internal estimates and the generative AI-derived narrative into a single response structure, selecting a disease name, a severity level, a numeric urgency index, and a recommendation text appropriate for display.

Server may append additional meta-information such as timestamps or an identifier of the model version used.

Input: Parsed generative AI outputs and internally calculated estimates including disease name, severity level, and urgency index.

Output: A structured response package containing all information needed by the terminal to present the assessment to the user.

### Step 13

Terminal receives the response package from the server via the communication interface.

Terminal parses the structured response to obtain the disease name, severity level, urgency percentage, and recommendation text, and stores these values temporarily in memory associated with the user interface.

Terminal updates display components such as text labels, progress bars, or colored indicators to show the estimated disease, severity, urgency, and recommended actions in a human-readable format.

Input: Structured response package from the server containing assessment results and recommendations.

Output: Updated on-screen presentation showing the health assessment outcome to the user.

### Step 14

User views the displayed information on the terminal and interprets the disease name, severity level, urgency percentage, and recommendation text.

User may decide to perform specific actions such as resting, hydrating, contacting a supervisor, or seeking immediate medical attention according to the recommended response content.

User may optionally use input controls on the terminal to submit feedback, such as indicating whether the assessment appeared correct or suggesting an alternative interpretation of the condition.

Input: Displayed assessment results and recommendations visible on the terminal.

Output: User actions in the real world, and optionally feedback data provided back to the system.

### Step 15

Server receives and processes user feedback sent from the terminal.

Server associates the feedback with the original feature vector, model outputs, and generative AI outputs stored in logs, and records this combined information as learning data for future model and prompt refinement.

Server may update weighting parameters for urgency calculation or adjust template components of the prompt sentence to better constrain the generative AI model in subsequent interactions.

Input: Feedback content from the user, together with identifiers linking the feedback to the previous assessment session.

Output: Updated configuration data, training samples, or adjusted prompt-generation rules that improve subsequent processing accuracy and efficiency.

It is also possible to incorporate an emotion engine for estimating the user's emotions. That is, the specific processing unit 290 may estimate the user's emotions using an emotion identification model 59, and perform specific processing based on the estimated emotions.

### Example 2

Description follows regarding a flow of the specific processing in an Example 2. The units of the system described below are implemented by the data processing device 12 and the smart device 14. The data processing device 12 is called a “server” and the smart device 14 is called a “terminal”.

Conventional computer-implemented health triage systems typically rely on static rule sets or pre-trained models that are executed on general-purpose processors. In such systems, the processor merely applies fixed threshold values or predetermined mappings from symptom inputs to urgency levels. As a result, even when large volumes of user interaction data and feedback data are accumulated in storage, the underlying triage logic executed by the processor is not adaptively refined in a systematic and automated manner. This leads to several technical problems from the perspective of computer technology.

First, the processor is not configured to dynamically construct context-rich analysis requests to an inference engine based on detailed, structured user state information. Instead, the processor often passes only coarse or hard-coded parameters to a model, which causes inefficient use of computing resources and limits the precision of the computed urgency scores and recommendation outputs. The internal data flow between the user interface, the storage subsystem, and the computation subsystem remains largely static and does not fully exploit available user-specific context in machine-interpretable form.

Second, the processor does not perform structured, feedback-driven updates of evaluation parameters or decision rules in a way that can be repeatedly executed as part of normal system operation. While some systems may allow human operators to manually tune rules or retrain models offline, such manual tuning is labor-intensive, non-scalable, and cannot respond in near real time to changes in user behavior or population-level patterns. From the viewpoint of computer architecture and system operation, this means the stored feedback data is not effectively converted, by machine processing, into refined control parameters for subsequent computation.

Third, existing systems generally lack a mechanism by which the processor automatically transforms accumulated input, output, and feedback triplets into machine-readable update instructions for an underlying inference engine. In many architectures, the processor simply logs the feedback and continues to execute the same inference algorithm without reconfiguring thresholds, weighting coefficients, or classification boundaries that govern emergency-level computation. Consequently, the data processing path from user input acquisition, through model inference, to feedback-based parameter update, is fragmented and not implemented as an integrated, computer-implemented optimization loop.

Fourth, in scenarios requiring high-urgency responses, such as determining when to notify an emergency response organization, known systems frequently perform only a basic threshold check on a single score and either trigger or do not trigger an alert. These systems do not exploit advanced, context-aware text generation mechanisms to assemble precise, machine-generated contact messages that reflect both the computed urgency and the detailed state information. This limits the quality and completeness of information transmitted by the processor to external entities and can degrade the overall efficiency of the emergency communication pipeline.

Accordingly, there is a need for a system-level improvement in the way a processor acquires, structures, and stores user state information; generates and supplies prompt sentences to a generative inference model; receives and interprets analysis results; and, importantly, uses user feedback and stored historical data to iteratively update evaluation parameters and decision rules. There is also a need for a processor-implemented mechanism that, upon determining that an emergency level satisfies a predefined condition, automatically generates and transmits context-rich contact messages to external emergency response organizations, while recording such transmissions as part of a coherent history. The technical problem addressed by the present invention is to improve the internal operation of the computer system-specifically, the configuration and coordination of the processor, storage device, information input/output device, and communication device-so that the system can automatically refine its triage logic and emergency communication behavior over time based on data-driven, feedback-driven processing.

The specific processing by the specific processing unit 290 of the data processing device 12 in Example 2 is realized by the following means.

The present invention provides a server comprising a processor, a storage device, an information input/output device, and a communication device, the processor being configured to acquire, via the information input/output device, input information from a user including state information comprising at least a cause, a symptom, and an elapsed time, to store the acquired input information as structured analysis data in the storage device, to generate, based on the structured analysis data stored in the storage device, a prompt sentence in which evaluation target information including the state information is embedded in a natural language format and/or a structured data format, to supply the generated prompt sentence to a generative AI model, to obtain from the generative AI model an analysis result including at least a health state, an abnormality severity, and an emergency level, to calculate, based on numerical information and/or classification information included in the analysis result, an evaluation index indicating a consultation urgency, to present the evaluation index and recommendation information obtained from the generative AI model to the user via the information input/output device, to store, in the storage device, the structured analysis data, the analysis result, and the evaluation index in association with one another as history information, to receive, via the information input/output device, feedback information from the user including evaluation information regarding at least one of the recommendation information and the consultation urgency, to store the feedback information in the storage device in association with the history information, to acquire, from the storage device, a plurality of case pieces of information each including a combination of the input information, the analysis result, and the feedback information, to generate an update prompt sentence that describes, in a natural language format, a learning context including the plurality of case pieces of information, to supply the update prompt sentence to the generative AI model to obtain a model response indicating update content of evaluation parameters and/or decision rules for calculation of the consultation urgency and generation of the recommendation information, to update, based on the update content included in the model response, threshold values, weighting coefficients, classification boundaries, or other control parameters used in consultation urgency calculation processing and recommendation information generation processing as setting information in the storage device, to execute subsequent analysis processing for new input information using the updated setting information, and, when determining, based on at least one of the analysis result and the updated setting information, that the consultation urgency is equal to or higher than a predetermined reference value, to generate a contact-sentence generation prompt sentence for generating contact content to an emergency response organization, to supply the contact-sentence generation prompt sentence to the generative AI model to obtain contact sentence information to be transmitted to the emergency response organization, to generate notification data including the contact sentence information and user identification information, to transmit the notification data to an external emergency response organization via the communication device, and to store a transmission history of the notification data in the storage device. This enables the computer system to implement, in a technically integrated manner, a feedback-driven optimization loop in which structured user state information and user feedback are converted into machine-interpretable prompts and parameter updates for the generative AI model, thereby dynamically improving the internal triage logic, the accuracy of computed consultation urgency indices, and the quality of automatically generated emergency contact messages while efficiently coordinating processing among the processor, storage device, information input/output device, and communication device.

The term “system” refers to an arrangement of hardware and software components including at least a processor, a storage device, an information input/output device, and a communication device, which cooperate to execute the claimed functions.

The term “processor” refers to a hardware processing unit, such as a central processing unit or other computing circuitry, configured to execute program instructions to perform the operations described in the claims.

The term “storage device” refers to a non-transitory computer-readable medium, such as a memory device or mass storage device, configured to store data, parameters, history information, and program instructions used by the processor.

The term “information input/output device” refers to one or more hardware interfaces, such as a display, a touch panel, a keyboard, a pointing device, a microphone, or a speaker, configured to present information to a user and to receive information from the user.

The term “communication device” refers to a hardware communication interface, such as a wired or wireless network interface, configured to transmit data to and receive data from external devices or systems over a communication network.

The term “user” refers to a human operator who provides input information to the system and receives output information, including evaluation results and recommendation information, via the information input/output device.

The term “input information” refers to data provided by the user, including at least state information such as a cause, a symptom, and an elapsed time, that is acquired by the processor via the information input/output device.

The term “state information” refers to information indicative of a current or past condition of a subject, including at least a cause that may influence the condition, a symptom representing a manifestation of the condition, and an elapsed time representing a duration from occurrence of the symptom.

The term “cause” refers to data indicating a factor, event, or condition that may have contributed to a health condition or abnormal state of a subject.

The term “symptom” refers to data indicating a perceived or observed manifestation of a health condition or abnormal state of a subject.

The term “elapsed time” refers to data indicating a length of time that has passed from a specified reference point, such as an onset of a symptom, to a current time or input time.

The term “structured analysis data” refers to input information that has been organized into a predetermined digital data structure, such as key-value pairs or records, suitable for storage, retrieval, and processing by the processor.

The term “evaluation target information” refers to data derived from the input information and state information that serve as a target for analysis by a model, including, for example, the cause, symptom, and elapsed time arranged in a form usable by the model.

The term “prompt sentence” refers to a text or data sequence, expressed in a natural language format and/or a structured data format, supplied to a model as an instruction or query to cause the model to generate an output.

The term “update prompt sentence” refers to a prompt sentence that describes a learning context including multiple case pieces of information, and that instructs a model to output update content for evaluation parameters and/or decision rules.

The term “contact-sentence generation prompt sentence” refers to a prompt sentence that instructs a model to generate contact content or a contact message intended for an emergency response organization.

The term “generative AI model” refers to a machine-implemented inference engine, such as a neural network or other parametric model, configured to generate output data, including text or structured data, in response to a prompt sentence.

The term “analysis result” refers to output data obtained from the generative AI model in response to a prompt sentence, including at least a health state, an abnormality severity, and an emergency level.

The term “health state” refers to data indicating an evaluated condition of a subject, such as stable, unstable, or other qualitative or quantitative representations of a health status.

The term “abnormality severity” refers to data indicating a degree of seriousness of an abnormal health condition, expressed, for example, as a score, level, or category.

The term “emergency level” refers to data indicating an urgency degree with which an action should be taken for a subject, such as a numeric score or classification representing how rapidly a response is required.

The term “evaluation index” refers to a computed value or set of values, derived from the analysis result, that quantitatively or qualitatively indicates a consultation urgency.

The term “consultation urgency” refers to a degree of necessity and rapidity for a subject to seek professional consultation, such as visiting a medical facility within a certain time frame.

The term “recommendation information” refers to data representing a proposed action or guidance for the user, generated based on the analysis result, such as advice to rest, consult a professional, or contact an emergency response organization.

The term “history information” refers to data stored in association, including structured analysis data, analysis results, evaluation indices, and related metadata, that can be referenced in subsequent processing.

The term “feedback information” refers to data received from the user indicating an evaluation of at least one of the recommendation information and the consultation urgency, such as ratings, comments, or qualitative assessments.

The term “case piece of information” refers to a record that includes, in combination, input information, an analysis result, and corresponding feedback information for a particular event or instance.

The term “learning context” refers to an aggregated description of one or more case pieces of information, arranged in a form suitable for use by the generative AI model to infer updates to evaluation parameters and/or decision rules.

The term “model response” refers to output data produced by the generative AI model in response to an update prompt sentence or other prompt sentence, including information describing update content for parameters, rules, or messages.

The term “evaluation parameters” refers to numerical or symbolic values used by the system to calculate the evaluation index, including, for example, threshold values, weighting coefficients, and classification boundaries.

The term “decision rules” refers to logical conditions, mappings, or rule sets used to determine an evaluation index, recommendation information, or control behavior of the system based on input data and analysis results.

The term “threshold values” refers to numerical boundary values used to categorize or compare computed scores, such as values that separate different ranges of consultation urgency.

The term “weighting coefficients” refers to numerical factors applied to one or more features or values when computing an evaluation index, to reflect the relative importance of those features.

The term “classification boundaries” refers to numerical or symbolic conditions that define divisions between different classes or categories in the evaluation of emergency level, consultation urgency, or other outputs.

The term “control parameters” refers to parameters, including threshold values, weighting coefficients, and classification boundaries, that govern the operation and output behavior of the system's evaluation and recommendation processing.

The term “setting information” refers to data stored in the storage device that defines current values of control parameters and other configuration data used by the processor in analysis and recommendation processing.

The term “emergency response organization” refers to an external entity or service, such as an emergency dispatch center or other response facility, that receives notification data and provides urgent assistance.

The term “contact sentence information” refers to text or structured data representing contact content or a message to be transmitted to an emergency response organization.

The term “notification data” refers to data generated for transmission to an external destination, including at least contact sentence information and user identification information.

The term “user identification information” refers to data that uniquely or sufficiently identifies a user or subject to an external system, such as an identifier, code, or other identifying data.

The term “transmission history” refers to data indicating details of notification data transmissions, including at least content identifiers, destinations, times, and statuses, stored in the storage device for recordkeeping and later reference.

In one embodiment, a server, a terminal, and a user cooperate to implement the claimed system. The server includes at least one processor, a main memory, a non-transitory storage device, an information input/output device such as a display and an operator console, and a communication device such as a wired or wireless network interface. The terminal includes a processor, a memory, a display, a touch panel, and a wireless communication module. The user operates the terminal to provide state information and receives evaluation results and recommendation information. The server executes a program stored in the storage device and loaded into the main memory to implement the functions described below.

The server uses a general-purpose operating system, such as a Unix-like server operating system, and an application framework, such as a web application framework, to implement an application layer program. The server uses a relational database management system, such as a database engine implementing SQL, as the storage device for structured analysis data, history information, feedback information, and setting information. The server also uses a generative AI model hosted either locally on the server or on another computing apparatus connected via the communication device. The generative AI model can be implemented as a transformer-based neural network, including a multi-layer architecture with self-attention layers, feedforward layers, and normalization layers, trained on a large corpus of domain-general and domain-specific text.

The server defines specific data structures in the storage device to store user-related information. For example, the storage device stores a table for user input events that includes fields such as: user identifier, cause, symptom, elapsed time, input timestamp, and additional metadata. The storage device stores a table for analysis results that includes fields such as: health state, abnormality severity score, emergency level score, recommendation text, and a foreign key linking to the corresponding input event. The storage device further stores a table for feedback information that includes fields such as: reference to an analysis result, feedback score, feedback comment, and feedback timestamp. The storage device also stores a table for control parameters and decision rules, including threshold values, weighting coefficients, classification boundaries, and rule descriptions used in consultation urgency calculation and recommendation generation.

The server uses the communication device to receive, from the terminal, structured analysis data corresponding to user input. The terminal runs an application, such as a mobile application implemented using a native application framework, that presents an input screen to the user. The terminal displays fields corresponding to “cause”, “symptom”, and “elapsed time” and other optional items. The user touches the screen and enters, for example, “unknown” as the cause, “continuous headache” as the symptom, and “24 hours” as the elapsed time. The terminal converts the user input into a structured data record and transmits the record to the server over a secure communication protocol.

The server receives the structured data record via the communication device and validates the content. The server checks that the elapsed time is within a reasonable numeric range, that the symptom field is non-empty, and that the user identifier is valid. The server then stores the validated data into the input event table in the database. By organizing the input information into a normalized database schema, the server enables efficient indexing, retrieval, and aggregation of state information for subsequent analysis and learning.

The server constructs, in the main memory, a prompt sentence directed to the generative AI model. The server embeds the cause, symptom, and elapsed time into a predefined prompt template. In one example, the server generates the following evaluation prompt sentence: “You are a medical triage generative AI model.

### Input Data

cause: unknown
symptom: continuous headache
elapsed time: 24 hours

Analyze this case and output a JSON-like structure with the following fields: health_state (string), abnormality_severity (0-100), emergency_level (0-100), recommendation (string).”

The server concatenates this prompt sentence as a single text sequence in the main memory. The server then sends the prompt sentence to the generative AI model via an application programming interface. When the generative AI model is deployed on the same physical machine, the server passes the prompt through a local inference library that runs the forward computation of the transformer network. When the generative AI model is provided as a remote service, the server transmits the prompt sentence over the communication device using an appropriate request protocol.

The server receives an analysis result in the form of a text response from the generative AI model. Internally, the generative AI model represents tokens of the prompt sentence as high-dimensional embeddings, propagates them through multiple self-attention layers, and computes output logits that are converted into probability distributions over tokens. The model generates output tokens sequentially, forming a text that includes, for example, a health state such as “unstable”, an abnormality severity such as “65”, an emergency level such as “70”, and a recommendation such as “Visit a clinic within the next few hours.” The server parses this text response according to a predefined grammar to extract numeric values and string fields. The server uses deterministic parsing logic, such as pattern matching or regular expressions, to robustly separate each field.

The server stores the analysis result fields in the analysis result table, linked to the original input event. The server then calculates, in the processor, an evaluation index indicating consultation urgency using a computation that combines the emergency level score, the abnormality severity score, and possibly other derived features. The server may use a weighted sum or a piecewise linear function, where the weighting coefficients and threshold values are stored as control parameters in the setting information table. By using structured parameters, the server can quickly adjust the mapping from analysis result values to consultation urgency indices without recompiling code.

The server uses the information input/output device to present the evaluation index and the recommendation information to the user, via the terminal. The server generates a response message that includes the evaluation index and recommendation text. The terminal receives this response and renders it as a graphical display, such as a colored bar indicating urgency and a text area describing the recommended action. The user can read, on the terminal, that the system recommends a clinic visit within a certain timeframe.

The user can provide feedback information to indicate whether the recommendation was appropriate. For example, after following the recommendation and receiving medical care, the user may input “The proposed response was quick and accurate” and select a feedback score of “5”. The terminal sends this feedback information to the server in structured form. The server receives the feedback, validates a reference to the corresponding analysis result, and stores the feedback in the feedback table. The server thereby accumulates a corpus of triplets including input information, analysis results, and feedback information.

The server periodically or on demand generates an update prompt sentence to refine evaluation parameters and decision rules. The server queries the database for a plurality of case records, each including input fields, analysis results, and feedback. The server encodes these cases into a text representation. In one example, the server constructs an update prompt sentence as follows: “You are an algorithm optimization generative AI model.

### Case 1

cause: unknown
symptom: continuous headache
elapsed time: 24 hours
system emergency_level: 70
system recommendation: ‘Visit a clinic within the next few hours.’
user feedback score: 5
user feedback comment: ‘The proposed response was quick and accurate.’

Based on this and similar cases, adjust the decision rules and threshold values for emergency_level and consultation urgency so that future recommendations for similar symptom patterns are more accurate.

Output updated numeric thresholds for low, medium, and high emergency_level, updated weighting coefficients for symptom duration, and brief textual rules describing when to increase or decrease the emergency_level.”

The server sends this update prompt sentence to the generative AI model. The generative AI model processes the prompt and outputs text specifying, for example, that low emergency level corresponds to scores 0-30, medium to 31-70, high to 71-100, and that for continuous headaches exceeding 24 hours with unknown cause, the emergency level should be increased by 10 points compared to previous logic. The server parses the output and updates the setting information in the database accordingly. The server writes new threshold values, weighting coefficients, and rule descriptions into the control parameter table.

Because the server stores the setting information in the database, the server can reload or cache the parameters into the main memory and apply them during subsequent evaluation processing. The server uses the updated threshold values and weighting coefficients when computing the evaluation index from new analysis results. This enables the system to dynamically refine its internal triage logic based on actual user feedback, in contrast to a static rule-based system.

The server also uses the generative AI model to generate contact sentence information for emergency response organizations. When the server determines that the consultation urgency index or emergency level satisfies a predetermined reference condition, the server constructs a contact-sentence generation prompt sentence that includes the current state information, the computed emergency level, and relevant user identifiers. The server may use, for example, the following prompt:
“You are a medical emergency communication assistant.
Generate a concise and clear contact message to be sent to an emergency response organization, based on the following data:
cause: unknown
symptom: continuous headache
elapsed time: 24 hours
emergency_level: 90
Include in the message the main symptom, the duration, the suspected severity, and a recommendation for immediate response.”

The server sends this prompt to the generative AI model, parses the resulting contact message, and constructs notification data including the contact sentence information and user identification information. The server transmits this notification data to an external emergency response organization using the communication device. The server also records a transmission history entry in the storage device, including a timestamp, destination identifier, and message summary.

From a technical perspective, the described configuration and processing provide improvements in computer technology beyond mere automation of human tasks. The server uses structured database schemas, numeric control parameters, and explicit prompt sentences to orchestrate a feedback-driven optimization loop. By storing control parameters separately from code and updating them based on model responses, the server reduces the need for manual reprogramming and enables rapid adjustment of computation logic. This leads to improved processing speed when adapting to new data patterns, because the server only modifies parameter values rather than retraining a full model or deploying new code.

Furthermore, the server uses the generative AI model to generate intermediate artifacts-such as updated thresholds and textual decision rules-that are explicitly parsed and converted into machine-readable parameters. This architecture allows the server to compress complex feedback patterns into low-dimensional parameter updates, thereby improving memory usage and database storage efficiency. Because the server aggregates multiple cases into a single update prompt, the communication load between the server and the model host is reduced compared to sending each case individually.

The generative AI model itself is implemented as a transformer-based neural network trained using supervised learning and, optionally, reinforcement learning from human feedback. During training, the model minimizes a loss function that includes cross-entropy between predicted and target tokens and, in some configurations, additional regularization terms. The model's parameters, such as weight matrices in attention heads and feedforward layers, are updated using gradient-based optimization algorithms. In some embodiments, the server fine-tunes an adapter module or a small subset of layers on domain-specific triage data, thereby reducing computational costs while improving domain accuracy.

The server exploits the generative AI model in a non-conventional manner. Rather than merely using the model to answer a single user query, the server repeatedly applies the model to both evaluation prompts and update prompts, and then uses the model's outputs to modify the system's internal decision logic. This cyclical interaction implements a closed feedback loop at the system level. As a result, the system can reduce error rates in emergency level estimation over time and can adapt to changing symptom distributions in a user population. The improved accuracy has a direct technical effect on downstream processes, such as reduction of false positive emergency notifications, which in turn reduces unnecessary network traffic and load on the communication device.

The use of structured features such as cause, symptom, and elapsed time, combined with explicit weighting and thresholding logic, enhances the interpretability and controllability of the system's behavior. By interpreting the generative AI model outputs as proposals for parameter changes, and by enforcing validation and range checks when updating parameters, the server maintains stability and predictability in its computations. This is different from conventional black-box approaches where a model directly outputs an urgency label without an explicit, adjustable parameter layer in between.

In alternative embodiments, the server can integrate additional features into the structured analysis data, such as demographic attributes or environmental conditions. The server can adjust the architecture of the generative AI model, for example by varying the number of attention layers or the size of the embedding vectors, to balance computational load and accuracy. The server can also employ a hybrid approach where a smaller local model handles routine cases using locally stored thresholds and rules, while a larger remote generative AI model is consulted only for complex or ambiguous cases. This variation further reduces communication latency and bandwidth usage while maintaining high precision for difficult cases.

In another embodiment, the server aggregates feedback information over a defined time window and computes statistical indicators, such as average feedback scores for particular symptom combinations. The server then incorporates these statistics into the update prompt sentences, allowing the generative AI model to generate more robust parameter updates. The server may also implement different update policies, such as applying parameter updates only if feedback scores exceed a threshold confidence level or if a sufficient number of cases have been accumulated.

Because the server explicitly organizes data, constructs prompt sentences, parses model outputs into control parameters, and updates internal logic in a structured way, the system achieves technical effects including improved triage accuracy, reduced processing time for future evaluations, better utilization of storage resources, and more efficient use of communication bandwidth. The integration of the generative AI model as a dynamically reconfigurable component within the computational pipeline, rather than as a fixed black-box classifier, constitutes an improvement in the functioning of the computer system as a whole.

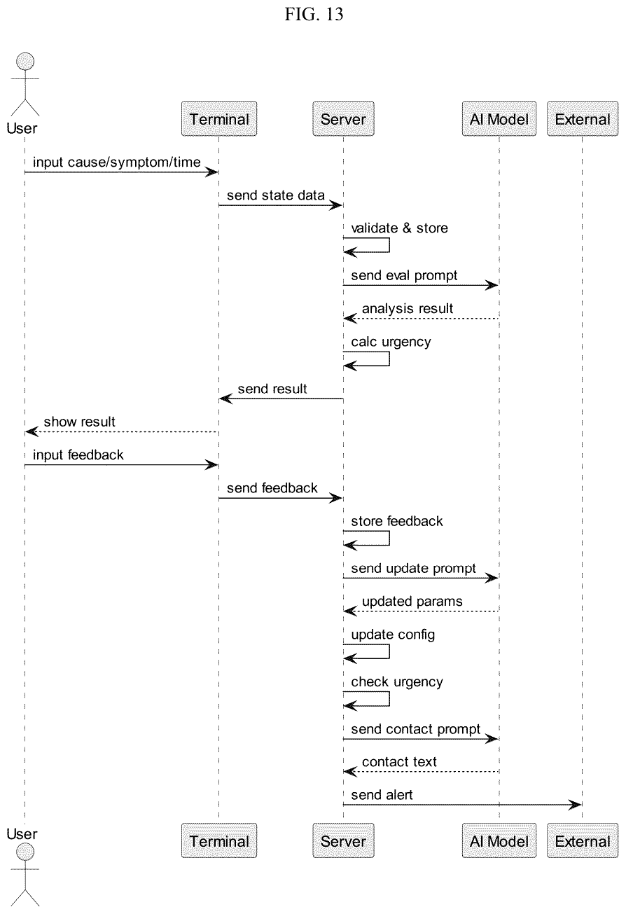

The following describes the processing flow using FIG. 13.

### Step 1

User operates the terminal to start a health-assessment application and to open an input screen.

User enters state information including at least a cause, a symptom, and an elapsed time by typing text and selecting items from lists.

Terminal receives this state information as input from the user interface, converts it into an internal data structure containing fields such as cause, symptom, elapsed_time, and user_id, and attaches a local timestamp.

Terminal outputs a structured record representing the user's state information, ready to be transmitted to the server.

### Step 2

Terminal takes the structured record as input, serializes it into a communication format such as key-value pairs, and prepares a network request containing the serialized data.

Terminal uses a communication module to send the network request to the server over a secure communication protocol.

Terminal outputs a request message including the state information, which is transmitted to the server.

### Step 3

Server receives the request message from the communication device as input.

Server parses the received data, extracts fields such as user_id, cause, symptom, elapsed_time, and timestamp, and validates the contents, for example by checking that elapsed_time is a numeric value within a permissible range and that mandatory fields are present.

Server performs data processing that includes type conversion, normalization of text strings, and assignment of default values for missing optional fields.

Server outputs a normalized and validated data object representing the user's state information.

### Step 4

Server takes the normalized data object as input and writes its fields into a persistent storage structure, such as an input event table in a relational database.

Server assigns a unique event identifier, stores the record, and commits the transaction to guarantee durability.

Server outputs an internal reference, such as the event identifier, and a stored record that can be retrieved for later analysis.

### Step 5

Server receives the stored record or the normalized data object as input and constructs a prompt sentence for a generative AI model.

Server embeds the cause, symptom, and elapsed_time values into a predefined text template to generate an evaluation prompt sentence.

For example, the server generates a prompt sentence such as: “You are a medical triage generative AI model.

### Input Data

cause: unknown
symptom: continuous headache
elapsed time: 24 hours

Analyze this case and output a JSON-like structure with the following fields: health_state (string), abnormality_severity (0-100), emergency_level (0-100), recommendation (string).”

Server combines the template and the embedded values to produce a single contiguous text string.

Server outputs the completed evaluation prompt sentence as the input for the generative AI model.

### Step 6

Server provides the evaluation prompt sentence as input to the generative AI model through an inference interface.

Server invokes a model API or a local inference engine, passing configuration parameters such as maximum output length and sampling settings.

Generative AI model processes the prompt sentence internally by converting tokens into embeddings, applying self-attention and feed-forward transformations, and generating output tokens that form an analysis result text.

Server receives the generated text as the output from the generative AI model.

### Step 7

Server takes the generated analysis result text as input and performs parsing operations.

Server applies pattern matching, tokenization, or a simple grammar to identify fields such as health_state, abnormality_severity, emergency_level, and recommendation.

Server converts numeric substrings into numeric types, checks that numeric values lie within expected ranges, and trims extraneous characters from text fields.

Server outputs a structured analysis result object containing, for example, a health_state label, an abnormality_severity score, an emergency_level score, and a recommendation text string.

### Step 8

Server receives the structured analysis result object and the control parameters from storage as input.

Server computes an evaluation index indicating consultation urgency by applying a predetermined algorithm, for example a weighted sum of emergency_level and abnormality_severity or a piecewise function using stored threshold values.

Server may normalize the computed index to a target scale, such as 0-100, by applying scaling factors.

Server outputs the evaluation index together with the analysis result object as processed evaluation data.

### Step 9

Server takes the processed evaluation data as input and records it in the database.

Server writes the analysis result fields and the evaluation index into an analysis result table, associating the entry with the corresponding input event via a foreign key.

Server thereby creates a linked history entry that combines input information, model output, and derived urgency data.

Server outputs stored history information that can be referenced in later feedback-driven updates.

### Step 10

Server generates a response message using the processed evaluation data as input.

Server formats the evaluation index and the recommendation text into a compact structure suitable for display, optionally including labels or severity categories derived from threshold comparisons.

Server sends this response message to the terminal via the communication device.

Server outputs a transmitted response containing the evaluation index and recommendation information.

### Step 11

Terminal receives the response message from the server as input.

Terminal parses the message, extracts the evaluation index, emergency_level, abnormality_severity, and recommendation text, and maps them to UI components.

Terminal performs display processing, such as converting the evaluation index into a color-coded bar and rendering the recommendation as multi-line text on the screen.

Terminal outputs a visual presentation on the display that the user can perceive.

### Step 12

User views the displayed evaluation index and recommendation on the terminal.

User evaluates whether the recommendation appears appropriate in view of actual symptoms and any subsequent medical outcome.

User operates the terminal to open a feedback input screen and enters a numerical feedback score and a free-text comment describing the evaluation of the recommendation.

User thereby outputs subjective feedback information to the terminal.

### Step 13

Terminal receives the feedback information from the user interface as input.

Terminal aggregates the feedback score, comment, and a reference to the corresponding analysis result or event identifier into a structured feedback record.

Terminal may add a local timestamp and device identifier, and then serializes the record into a communication format.

Terminal outputs a feedback request message and transmits it to the server using the communication module.

### Step 14

Server receives the feedback request message through the communication device as input.

Server parses the message, validates the association with a stored analysis result, and checks that the feedback score is within a permitted numeric range.

Server writes the validated feedback data into a feedback table in the database, linking it to the relevant history entry.

Server outputs an updated history set that now includes feedback information associated with the original input and analysis result.

### Step 15

Server collects, from the storage device, a plurality of history entries and corresponding feedback records as input for parameter updating.

Server selects representative cases based on criteria such as symptom type, elapsed_time range, or recency, and builds a text description of each case including input fields, analysis results, and feedback content.

Server then constructs an update prompt sentence that embeds multiple such case descriptions into a coherent learning context for the generative AI model.

Server outputs the completed update prompt sentence to be used in model-assisted parameter refinement.

### Step 16

Server supplies the update prompt sentence as input to the generative AI model using the same or a dedicated inference interface.

Generative AI model processes the update prompt sentence, internally correlating input patterns, output errors suggested by feedback scores, and adjustment directions.

Generative AI model generates text describing updated threshold values, weighting coefficients, and qualitative rule modifications, such as increasing urgency for prolonged symptoms with poor feedback history.

Server receives this text as the model response output.

### Step 17

Server uses the model response text as input and performs parsing operations to extract numeric and symbolic update content.

Server identifies proposed new thresholds for low, medium, and high emergency_level, new weighting coefficients for features such as elapsed_time and specific symptom categories, and structured rule notes indicating conditions for adjustments.

Server checks each proposed parameter against safety and range constraints, discarding or clamping values that fall outside predetermined bounds.

Server outputs a validated set of updated control parameters and rule descriptions.

### Step 18

Server takes the validated control parameters as input and updates the setting information in the storage device.

Server writes new threshold values, weighting coefficients, classification boundaries, and rule notes into the control parameter table, replacing or versioning older values.

Server may also load the updated parameters into in-memory data structures to avoid repeated database access during subsequent evaluations.

Server outputs an updated configuration state that will be applied to future consultation urgency calculations and recommendation generation.

### Step 19

Server applies the updated configuration state as input to later analysis cycles.

When new state information arrives from the terminal, server uses the updated control parameters while calculating the evaluation index from the analysis result, thereby modifying the mapping from model output to urgency categories.

Server implicitly incorporates historical feedback into each new evaluation through the modified thresholds and weights, thereby changing how the processor computes results in response to similar input patterns.

Server outputs refined evaluation indices and recommendations that reflect the feedback-driven adjustments.

### Step 20

Server monitors newly computed evaluation indices and emergency levels as input for emergency handling.

When server determines that an evaluation index or emergency_level meets or exceeds a predetermined reference value, server composes a contact-sentence generation prompt sentence including the current state information, computed urgency values, and necessary identification data.

Server sends this contact-sentence generation prompt sentence to the generative AI model to obtain contact sentence information tailored for an emergency response organization.

Server outputs the generated contact message, encapsulates it into notification data together with user identification information, and transmits the notification data to an external emergency response organization via the communication device, while also recording a transmission history entry in the storage device.

### Application Example 2

Description follows regarding a flow of the specific processing in an Application Example 2. The units of the system described below are implemented by the data processing device 12 and the smart device 14. The data processing device 12 is called a “server” and the smart device 14 is called a “terminal”.

Conventional computer-implemented triage and recommendation systems generally treat user input, biometric sensing, and emotional context as separate, weakly coupled streams. In many deployed architectures, a server receives simple symptom text via a user interface, executes a fixed ruleset or a static machine learning model, and returns a single urgency score or recommendation. Such systems exhibit several technical shortcomings.

First, conventional systems are typically limited to shallow pattern matching on raw text and basic thresholding on biometric values. They do not leverage a unified representation of heterogeneous data types-such as structured “cause, symptom, elapsed time” fields, continuous vital-sign data, and emotion signals-from which a machine can compute a more discriminative feature space for triage. As a result, model inputs are noisy and poorly normalized, which degrades the quality of inferences, increases misclassification rates for urgent vs. non-urgent cases, and forces conservative thresholds that generate many false positives or false negatives. From a computer-technology standpoint, this reflects an inefficient utilization of available sensor data and natural language information in the server's decision pipeline.

Second, most existing systems do not use generative AI models in a controlled, machine-oriented manner. When a generative model is used at all, it is often prompted ad hoc with natural language instructions and returns free-form text that is hard for the server to parse reliably. This leads to brittle downstream processing, inconsistent output formats, and a need for extensive rule-based post-processing, which increases latency and computational overhead. The absence of a standardized prompt sentence structure and structured AI output restricts the ability of the processor to integrate generative reasoning into real-time triage logic.

Third, conventional architectures generally treat user feedback only as an informal signal, if they store it at all. Feedback is typically not converted into structured training data and is not used to automatically adjust recommendation algorithms or generative model behavior. Consequently, the system cannot systematically reduce its own error patterns or adapt to population-specific or user-specific characteristics. From the perspective of computer technology, this means that the server operates with essentially static models that cannot efficiently close the loop between prediction, outcome, and online learning, resulting in suboptimal resource usage and persistent bias.

Fourth, emergency escalation mechanisms are frequently implemented as simple threshold triggers that operate on one modality (for example, heart rate). They do not integrate emotion analysis or generative planning of emergency messages. Thus, when vital signs and emotional states jointly indicate high risk, the server is not able to synthesize rich, context-aware emergency contact content in a machine-automated way, and must rely on prewritten templates. This limits the expressiveness and relevance of notifications sent to emergency responders and may require additional human intervention to interpret the situation.

Accordingly, there is a need for improved computer-implemented techniques and server architectures that: (i) integrate situation information, biometric information, and emotion information into unified feature data; (ii) generate structured prompt sentences that drive a generative AI model to produce machine-parseable analysis results; (iii) automatically compute and adjust visit urgency or emergency indices based on those results; (iv) use structured user feedback and AI-based feedback analysis to update model parameters; and (v) automatically generate and transmit context-rich emergency messages when thresholds are exceeded. Such techniques should improve the technical functioning of the triage and alert system itself by reducing parsing failures, improving model accuracy, lowering false alarm rates, and enabling efficient, automatic adaptation of the underlying computational models.

The specific processing by the specific processing unit 290 of the data processing device 12 in Application Example 2 is realized by the following means.

The present invention provides a server comprising a processor configured to provide a user interface that receives situation information including at least a cause, a symptom, and an elapsed time from a terminal device, to acquire biometric information and emotion information from the terminal device, to preprocess the situation information, the biometric information, and the emotion information into feature data, to generate a prompt sentence that instructs a generative AI model to perform analysis using the feature data, to input the prompt sentence into the generative AI model and obtain an analysis result, to calculate, on the basis of the analysis result and the feature data, a visit urgency or an emergency index, to generate response content including at least a recommendation for an action according to the visit urgency or the emergency index and cause the user interface to display the response content, to acquire evaluation information including user feedback, to generate a prompt sentence that instructs the generative AI model to analyze the evaluation information and obtain a feedback analysis result, to update one or more parameters of at least one of the generative AI model and an algorithm for calculating the visit urgency or the emergency index on the basis of the feedback analysis result, and to, when the visit urgency or the emergency index exceeds a predetermined threshold, generate a prompt sentence that instructs the generative AI model to generate emergency contact content, obtain the emergency contact content, create notification data including at least the emergency contact content, location information, and the biometric information, and automatically transmit the notification data to an emergency response organization via a communication function. This enables the server to technically improve the processing of heterogeneous health-related data by unifying situation, biometric, and emotion inputs into machine-usable feature data, to reliably exploit a generative AI model through structured prompt sentences and structured outputs, to compute more accurate and adaptive urgency indices, to iteratively refine internal models based on structured feedback analysis, and to automatically synthesize and dispatch context-rich emergency notifications, thereby enhancing the overall computational performance, robustness, and responsiveness of the triage and alert system.

The term “system” refers to a combination of hardware and software components including at least one processor, memory, communication interface, and program code that cooperatively perform the functions described in the claims.

The term “processor” refers to a hardware processing circuit, such as a central processing unit, a microcontroller, or a hardware logic device, that executes instructions to perform arithmetic, logical, and control operations.

The term “user interface” refers to a logical interface presented to a user via a display device and input device, enabling the user to input information and to receive output, and may include graphical user interfaces, text-based interfaces, or voice-based interfaces.

The term “terminal device” refers to an end-user computing device operated by a user, such as a smartphone, a tablet, a wearable device, or a personal computer, that communicates with the server and captures user input, biometric information, and emotion information.

The term “situation information” refers to information describing a current health-related or event-related context of a user, including at least a cause, a symptom, and an elapsed time associated with the cause or the symptom.

The term “cause” refers to information indicating an origin, trigger, or contributing factor associated with a condition or event experienced by a user.

The term “symptom” refers to information indicating a perceived abnormal state, sign, or complaint experienced by a user, including physical or psychological manifestations.

The term “elapsed time” refers to a duration between a reference point, such as onset of a symptom or occurrence of a cause, and a current time, represented as a time value or time period.

The term “biometric information” refers to data representing physiological characteristics of a user, including but not limited to heart rate, blood pressure, body temperature, and other vital sign measurements.

The term “emotion information” refers to data indicating a psychological or emotional state of a user, such as anxiety, calmness, panic, relief, or satisfaction, derived from user input, voice, facial expression, text, or other signals.

The term “feature data” refers to structured data obtained by preprocessing and transforming situation information, biometric information, and emotion information into numerical or categorical values suitable as input for a machine-learning model or a generative AI model.

The term “preprocessing” refers to one or more operations applied to raw input data, including normalization, tokenization, encoding, filtering, and aggregation, to produce feature data for subsequent analysis.

The term “generative AI model” refers to an artificial intelligence model configured to generate output data, such as natural language text or structured content, in response to input data or instructions, using learned statistical patterns.

The term “prompt sentence” refers to an instruction text provided as input to the generative AI model, specifying a task, input conditions, and desired output format, and guiding the generative AI model in producing an analysis result or contact content.

The term “analysis result” refers to output data produced by the generative AI model in response to a prompt sentence, including at least an interpretation of feature data, an estimated condition, or an evaluation relevant to urgency or recommendation.

The term “visit urgency” refers to a numerical or categorical indicator representing a degree of necessity or priority for a user to visit a medical institution, computed on the basis of the analysis result and the feature data.

The term “emergency index” refers to a numerical measure indicating a level of emergency associated with a user's condition or situation, computed from feature data and analysis results, and used for determining whether an emergency response is required.

The term “response content” refers to output information generated by the processor, including at least a recommendation for an action, explanatory text, or other guidance presented to the user via the user interface.

The term “evaluation information” refers to feedback data provided by a user, including ratings, comments, or other indications concerning appropriateness or usefulness of previous recommendations or system behavior.

The term “feedback analysis result” refers to structured information obtained by analyzing evaluation information, including at least an inferred emotion state, an assessment of recommendation appropriateness, or extracted keywords.

The term “parameter” refers to a numerical value, weight, threshold, or configuration setting that influences behavior of a generative AI model, a machine-learning model, or an urgency calculation algorithm.

The term “algorithm for calculating the visit urgency or the emergency index” refers to a computational procedure, which may include rule-based logic, statistical methods, or machine-learning inference, that outputs a visit urgency or emergency index from feature data and analysis results.

The term “emergency contact content” refers to information describing an emergency situation and requested assistance, generated for transmission to an emergency response organization, and including at least a summary of the user's condition.

The term “notification data” refers to data transmitted to an external entity, such as an emergency response organization, including at least emergency contact content, location information, and biometric information.

The term “location information” refers to data identifying a geographic position associated with a user or terminal device, such as coordinates, address information, or other positional descriptors.

The term “emergency response organization” refers to an entity that provides emergency assistance, such as an emergency dispatch center, a medical emergency service, or another public safety organization.

The term “communication function” refers to a hardware and software capability that enables data transmission between the server, terminal device, and external entities over a communication network, and may include wired or wireless communication protocols.

In one embodiment, a server, one or more terminals, and one or more external communication services cooperate to implement the claimed system. The server comprises at least one processor, a memory storing program code and data structures, and a communication interface. The terminal comprises at least one processor, a user interface device such as a touchscreen display and microphone, a communication interface such as a wireless transceiver, and optionally one or more sensors or interfaces to wearable sensors.

The server executes an application program on a general-purpose computing platform. The server may use a software stack including an operating system, a web framework such as a generic server framework, a scripting language runtime such as a general-purpose interpreted language, and data analysis libraries such as a tabular-data processing library and a numerical-computation library. The server further stores and executes one or more machine-learning models and a generative AI model. The generative AI model may be a large language model deployed on the server or accessed via an external model service through an application programming interface.

The terminal executes a client application on a mobile operating system or desktop operating system. The terminal application provides a user interface for inputting situation information and feedback, and for displaying results. The terminal may be a smartphone, a tablet, a wearable device, or a personal computer. The terminal may integrate with a wearable sensor platform, a physiology-data collection interface, or an equivalent platform to acquire biometric information such as heart rate, blood pressure, and body temperature. The terminal may also include or access an emotion-detection module that analyzes user voice or facial images locally, or the terminal may capture raw audio or video and upload it to the server for server-side emotion analysis.

In one implementation, the user operates the terminal to launch the client application and views a user interface screen. The terminal displays input fields for a cause, a symptom, and an elapsed time. The terminal allows the user to enter free-form text for these fields and may also provide selectable options. The terminal further provides controls for the user to consent to use of biometric information and emotion information. When the user grants consent, the terminal reads biometric information from a connected wearable device and may acquire audio or video samples for emotion analysis.

The terminal transmits situation information, biometric information, and optionally raw or pre-computed emotion information to the server through a network, using a communication protocol such as HTTPS. The terminal structures the data using a predefined data schema so that the server can correctly associate cause, symptom, elapsed time, biometric values, and emotion indicators with a particular user and context.

The server stores program code that defines a data-processing pipeline in memory. The server loads received situation information, biometric information, and emotion information into the data-processing pipeline. The server uses a data-frame based processing library to normalize and transform the received information into feature data. For example, the server converts textual cause and symptom fields into token sequences using a tokenizer, maps tokens to integer indices using a vocabulary table, and constructs embeddings for each token using an embedding matrix. The server converts elapsed time from a textual representation such as “2 hours” into a numeric scalar such as 120 minutes. The server normalizes biometric values such as heart rate and blood pressure by subtracting a population mean and dividing by a standard deviation, thereby obtaining standardized features. The server converts categorical or discrete emotion labels such as “panic” or “relief” into one-hot encodings or continuous scores, and the server may also derive intensity values from underlying emotion analysis models.

The server concatenates or otherwise combines these normalized elements into feature vectors. In one embodiment, the server constructs a multi-modal feature representation in which textual features, biometric features, and emotion features are organized into separate sub-vectors and then merged through a linear transformation or attention mechanism in a neural network. This structured feature data forms an input suitable for both discriminative models (such as feed-forward neural networks) and for constructing prompt sentences for a generative AI model.

The server maintains a generative AI model that is capable of processing prompt-based natural language and returning an analysis result. The generative AI model may be implemented as a transformer-based neural network having an encoder-decoder structure or a decoder-only structure. The model may include multiple layers of self-attention, feed-forward sublayers, layer normalization, and positional encodings. The server may host this model locally in memory or access an external generative AI model through an application programming interface. During inference, the model receives a prompt sentence that includes instructions, context, and optionally a requested output format.

The server generates a prompt sentence in natural language that encodes the feature data in a structured manner. For example, the server may generate a prompt sentence such as: “You are a medical triage assistant. Based on the following information, infer the most likely condition, estimate severity as low, medium, or high, and assign a visit-urgency percentage (0-100). Then provide a short explanation and an action recommendation. Information: cause: ‘sudden severe headache’; symptom: ‘vomiting’; elapsed time: ‘2 hours’; heart rate: 110; blood pressure: ‘ 140/90’; emotion: ‘anxious’.”

The server may also generate prompt sentences for analyzing user feedback. For example, the server may generate a prompt sentence such as: “Analyze the following user feedback on a previous recommendation. Determine whether the user considered the advice appropriate (yes or no), infer the dominant emotion, and list three keywords that describe the feedback. Feedback: ‘The advice was correct; I felt reassured.’ Return the result in a structured textual form that can be easily parsed.”

The server encodes the prompt sentence using a tokenizer and sends the tokenized prompt to the generative AI model. The generative AI model performs internal computations, such as computing self-attention over the prompt tokens, applying learned weights to derive contextual embeddings, and generating output tokens one by one according to a probability distribution defined by softmax over the vocabulary. The model's weights have been trained or fine-tuned on medical and conversational text data using a supervised or unsupervised learning method, such as next-token prediction or instruction-following learning. During training, loss functions such as cross-entropy are minimized using gradient-based optimization methods such as stochastic gradient descent or an adaptive gradient method, and weights are updated by back-propagation.

The server receives the output tokens from the generative AI model and decodes them into text. The server may constrain the output using techniques such as temperature adjustment, nucleus sampling, or beam search to reduce randomness and promote structured outputs. The server then parses the generative AI model's output. In one embodiment, the server requests that the model provide explicit labels such as “condition: . . . ”, “severity: . . . ”, “visit-urgency: . . . ”, and “recommendation: . . . ” so that the server can reliably identify and extract each element using pattern matching or simple parsing rules. This approach avoids brittle free-form text handling and thereby improves computational robustness.

In order to compute a visit urgency or emergency index, the server may use a hybrid approach. The server may derive an initial urgency estimation from the generative AI model's output and then adjust the estimation using an additional discriminative model. For example, the server may implement a feed-forward neural network that accepts as input a vector composed of numeric features and encoded categories from the analysis result and the feature data. The network may have several hidden layers with non-linear activation functions such as rectified linear units. The network outputs a scalar value representing a refined urgency index. The server may use a loss function such as mean squared error between predicted urgency and reference urgency (obtained from historical labels or expert annotations) during training. The server updates network weights using gradient descent and regularization techniques to prevent overfitting.

By combining the generative AI model's semantic understanding with a discriminative model tuned to urgency prediction, the server can improve accuracy and reduce misclassification error compared to either a purely rule-based system or a single model that does not specifically account for the different modalities. The server may also encode non-linear interactions between biometric anomalies and emotion states, such as giving additional weight to “panic” when vital signs are borderline, and thereby produce more nuanced urgency indices.

The server generates response content for the user. The response content includes at least the inferred condition, the severity level, the computed visit urgency or emergency index, and recommended actions. The server formats the response content as structured data and transmits it to the terminal. The terminal receives the structured data, parses it, and updates the user interface. The terminal may show, for example, a message such as “There is a high probability of migraine. Visit urgency is 80%. It is recommended to visit an emergency clinic as soon as possible.” The terminal may also render a graphical representation of the urgency, such as a color-coded bar. This visual encoding of urgency allows rapid human interpretation, but the underlying numeric representation also allows consistent threshold comparison inside computer logic.

The server further operates to acquire evaluation information including user feedback regarding previous recommendations. The user interacts with the terminal to rate recommendations and to enter comments describing their experience. The terminal transmits this evaluation information to the server. The server stores evaluation information in a database and later processes it in batches or streams.

For feedback analysis, the server again uses the generative AI model, but now with a prompt sentence tailored to feedback. For instance, the server may use prompt sentences such as: “Analyze the following feedback about a health recommendation. Determine if the user agreed with the urgency (yes or no), infer the primary emotion (for example, ‘relieved’, ‘still anxious’, ‘angry’), and identify any mention of delay or misjudgment. Feedback: ‘The urgency seemed too low; I had to go to the hospital later.’”

The server uses the generative AI model to transform free-form feedback into structured feedback analysis results. The server extracts labels such as “appropriateness,” “primary emotion,” and “key issues.” The server then uses these labels to create training examples for updating the urgency-prediction network and any additional models. This enables the server to adjust internal parameters based on real-world outcomes, rather than relying solely on static expert rules.

In an emergency context, the server uses both live biometric information and emotion information to determine whether to trigger an emergency response. The terminal reads real-time vital signs from a wearable sensor platform and periodically transmits the data to the server. The server compares these data against stored baseline values and thresholds. For example, the server may maintain threshold ranges for heart rate and blood pressure, and may also apply temporal filters such as moving averages to reduce noise. The server also receives emotion information from an emotion-recognition module. When both biometric features and emotion features indicate a high-risk state, the server increases an emergency index.

When the server determines that the emergency index exceeds a predetermined threshold, the server generates a prompt sentence for the generative AI model to generate emergency contact content. For example, the server may use a prompt sentence such as: “Compose a concise emergency message for an emergency response organization. The message should include key symptoms, recent vital signs, and a clear request for urgent assistance. Information: cause: ‘sudden chest pain’; symptom: ‘shortness of breath’; elapsed time: ‘30 minutes’; heart rate: 182; blood pressure: ‘ 150/95’; emotion: ‘panic’. Format the message as a short paragraph suitable for an operator script.”

The server receives emergency contact content from the generative AI model and then constructs notification data that includes the emergency contact content, current location information from the terminal, and the latest biometric information. The server transmits the notification data to an emergency response organization via the communication interface. In some embodiments, the server interacts with a telephony or messaging gateway service to place an automated voice call or to send a text message containing the emergency contact content.

By automatically combining live sensor data, emotion analysis, and generative composition of emergency messages, the server reduces the time required to prepare accurate and context-rich notifications. This reduces cognitive load on human operators and decreases the likelihood of missing critical information, thereby providing a technical improvement over simple threshold-triggered messages that contain only a numeric value or short template.

The described structure yields specific technical effects. The server's feature-extraction pipeline and multi-modal feature representation improve data quality and model input consistency, which in turn improve machine inference accuracy. The use of structured prompt sentences for the generative AI model reduces parsing errors and enables reliable, machine-parseable responses, which improves processing speed and reduces computational overhead required for complex post-processing. The feedback-driven parameter-update mechanism allows the models to adapt to real-world usage without manual re-programming, reducing maintenance effort and improving long-term accuracy. The integration of emotion and biometric information in emergency detection improves sensitivity and specificity, which reduces false alarms and missed emergencies, and thus improves overall system reliability.

Furthermore, the server does not merely automate a human decision-making process; rather, the server implements specialized internal data structures and non-conventional processing flows that are not naturally performed by humans. For example, the server maintains high-dimensional feature vectors derived from tokenized text, standardized physiological measures, and encoded emotion states, and uses these vectors in neural network computations that involve large matrix multiplications and attention operations. The server uses dynamic prompt generation with explicit output-format constraints to interface with a generative AI model in a way that optimizes computer-internal processing. These steps are directed to improving the functioning of the triage and alert computer system itself, for example by reducing processing latency and error rates in computing urgency indices and in generating emergency messages.

Alternative embodiments can vary in architecture while still implementing the claimed invention. In one variation, the generative AI model resides entirely within the server, and the server loads a pre-trained transformer model into memory for local inference, thus eliminating dependency on external services and reducing network latency. In another variation, the generative AI model is accessed as an external service, and the server optimizes prompt length and content to reduce communication bandwidth while maintaining sufficient context for accurate analysis. In a further variation, the system may use different neural network architectures for urgency prediction, such as recurrent neural networks, convolutional neural networks, or graph neural networks, as long as the server continues to preprocess situation, biometric, and emotion information into feature data and to calculate urgency indices based on those features and generative outputs.

The terminal may also vary in configuration. In one embodiment, the terminal performs local emotion analysis using an on-device model to reduce network usage and improve privacy. In another embodiment, the terminal simply captures raw audio and image data and transmits them securely to the server, which then performs emotion analysis using more computationally intensive models. Regardless of these variations, the core functions of receiving situation information, transmitting structured data to the server, and displaying server-generated response content remain.

The user's role remains consistent across embodiments. The user operates the terminal to enter situation information, observes recommendations and urgency indicators, and provides feedback about the quality of recommendations. However, the technical improvements described reside primarily in the server and terminal processing architecture, which defines how data is represented, how the generative AI model and other models are orchestrated, how feedback is transformed into parameter updates, and how emergency notifications are automatically generated and transmitted. These aspects collectively provide a concrete technological solution to the problem of robust, adaptive, and accurate health-related triage and emergency alerting in a computer-implemented system.

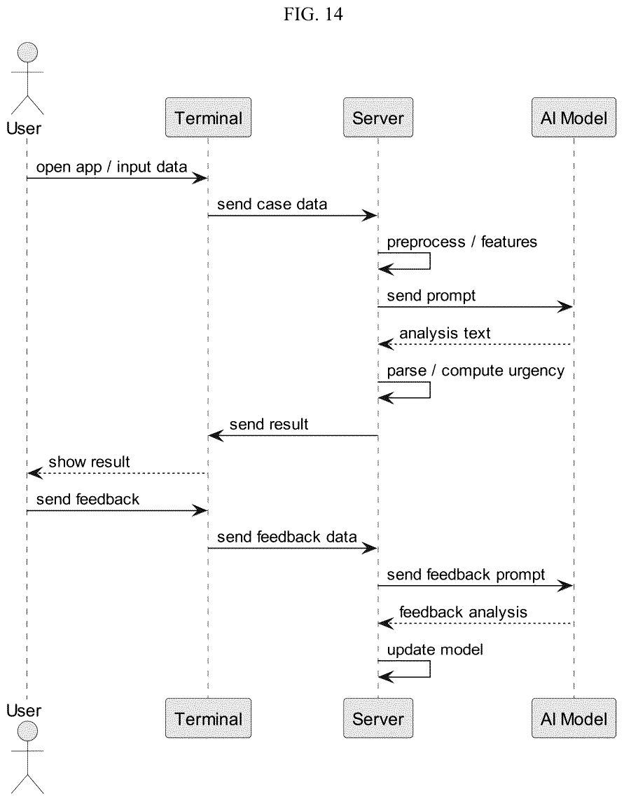

The following describes the processing flow using FIG. 14.

### Step 1

User operates the terminal to start a client application and open an input screen.

User inputs situation information including at least a cause, a symptom, and an elapsed time into text fields or selects from predefined options displayed on the terminal.

Input: raw user keystrokes or selections for cause, symptom, and elapsed time.

Output: structured situation information stored in the terminal's local data structure (for example, a key-value map containing cause, symptom, elapsed time).

### Step 2

Terminal acquires biometric information from connected sensors or a wearable platform and optionally acquires emotion information.

Terminal communicates with a sensor interface or a health-data platform to read values such as heart rate, blood pressure, and temperature, and may also read pre-computed emotion labels from an on-device emotion module.

Input: sensor signals or health-platform API responses, and optional emotion labels.

Output: a biometric-emotion data object containing numeric vital signs and emotion indicators associated with the user.

### Step 3

Terminal combines the situation information and the biometric-emotion data into a request payload and prepares it for transmission.

Terminal serializes the combined data into a structured format such as JSON, attaches a user identifier and timestamp, and performs basic validation checks (for example, non-empty fields, numeric ranges for vital signs).

Input: structured situation information and biometric-emotion data.

Output: a validated request payload ready to be sent to the server through a network interface.

### Step 4

Terminal transmits the request payload to the server via a secure communication channel.

Terminal opens an HTTPS connection to a predetermined server endpoint and sends the payload as the body of an HTTP request, then waits for a response.

Input: validated request payload and server endpoint information.

Output: a network message delivered to the server containing situation information, biometric information, and emotion information.

### Step 5

Server receives the network message and parses the payload into internal data structures.

Server reads the HTTP request, decodes the structured format, verifies the presence of required fields, and logs the raw data for auditing.

Input: network message containing the request payload.

Output: internal representations of situation information, biometric information, and emotion information in memory.

### Step 6

Server preprocesses the situation information, biometric information, and emotion information into feature data.

Server tokenizes text fields, maps tokens to integer indices using a vocabulary, converts elapsed time to a numeric value, normalizes vital signs using stored means and standard deviations, and encodes emotion labels into vectors.

Input: raw text values for cause, symptom, elapsed time, raw vital-sign values, and raw emotion indicators.

Output: a feature vector or set of feature vectors that numerically represent the multimodal input.

### Step 7

Server generates a prompt sentence for a generative AI model using the feature data.

Server inserts the normalized values and categorical encodings into a natural-language template that specifies the analysis task and desired output format.

Input: feature data and a stored prompt template.

Output: a prompt sentence in natural language describing the user's situation, vital signs, emotion, and requested analysis.

### Step 8

Server transmits the prompt sentence to the generative AI model and obtains an analysis result.

Server encodes the prompt into tokens, sends these tokens to the generative AI model through a local inference engine or a remote model service, and receives generated tokens as the model's response.

Input: prompt sentence representing the user's case.

Output: generated text containing an analysis result, such as an estimated condition, severity level, urgency suggestion, and explanation.

### Step 9

Server parses the analysis result into structured elements usable by downstream computation.

Server scans the generated text for labeled fields or patterns, extracts values such as condition name, severity category, and suggested urgency, and converts them into machine-readable forms (for example, numeric severity codes and urgency estimates).

Input: free-form or semi-structured analysis text from the generative AI model.

Output: structured analysis data including condition, severity, initial urgency, and rationale tokens.

### Step 10

Server computes a final visit urgency or emergency index using the feature data and the structured analysis data.

Server inputs both the feature vector and the structured analysis data into a predictive algorithm, which may be a neural network or rule-based logic, and performs numeric operations such as matrix multiplications and non-linear activations to output a refined urgency score.

Input: feature data and structured analysis data.

Output: a numerical urgency value (visit urgency or emergency index) within a defined range and an associated classification (for example, low, medium, high).

### Step 11

Server generates response content for presentation to the user.

Server composes a message that includes the estimated condition, severity level, urgency value, and recommended actions, and packages this message together with any additional information such as explanatory text or follow-up suggestions.

Input: final urgency value, condition estimate, and recommendation rules or templates.

Output: response content structured for transmission to the terminal, including text strings and associated numeric indicators.

### Step 12

Server checks whether the computed urgency or emergency index exceeds a predetermined threshold and, if so, prepares emergency contact content.

Server compares the urgency value against one or more stored thresholds, and when a threshold is exceeded, the server generates or refines an emergency contact description, which may involve an additional prompt sentence to the generative AI model to produce concise contact content.

Input: final urgency or emergency index and threshold values.

Output: a decision flag indicating whether emergency escalation is required and emergency contact content if escalation is triggered.

### Step 13

Server creates notification data for an emergency response organization when escalation is required.

Server aggregates emergency contact content, latest biometric values, and location information received from the terminal, and encodes them into a structured notification suitable for telephony or messaging services.

Input: emergency contact content, biometric information, location information, and escalation decision flag.

Output: a complete notification data object ready for transmission to an external emergency response endpoint.

### Step 14

Server transmits the notification data to an emergency response organization via a communication function.

Server connects to a communication gateway or network service, issues a call or message request that includes the notification data, and receives confirmation or status information from the external service.

Input: notification data and communication endpoint configuration.

Output: an emergency call initiation or emergency message sent to the emergency response organization and a status response stored in the server.

### Step 15

Server constructs a response payload for the terminal including either standard triage content, emergency-escalation information, or both.

Server merges the response content created for the user with any additional status messages about emergency calls and formats the combined information into a structured payload for the terminal.

Input: response content for the user and status or escalation information.

Output: a structured response payload containing triage results and, if applicable, emergency call status.

### Step 16

Terminal receives the response payload from the server and updates the user interface.

Terminal parses the structured response, displays the condition estimate, severity, urgency value, and recommendations, and, if emergency escalation occurred, shows an indication that an emergency contact has been made.

Input: structured response payload received over the network.

Output: updated visual and optional audio output on the terminal, presenting triage results and emergency status to the user.

### Step 17

User observes the displayed information and, after some time, provides feedback on the recommendation quality.

User interacts with a feedback screen on the terminal to select a rating and optionally enters free-form comments reflecting satisfaction or dissatisfaction with the prior advice.

Input: perceived experience of the user regarding the previous triage and displayed recommendations.

Output: raw feedback signals (rating values and text comments) captured by the terminal.

### Step 18

Terminal packages the feedback into a structured feedback payload and sends it to the server.

Terminal associates the feedback with a prior case identifier, formats the rating and comment into structured fields, and transmits them via a network request.

Input: raw rating and textual comment from the user.

Output: a feedback payload delivered to the server, containing evaluation information linked to a specific triage case.

### Step 19

Server receives the feedback payload and converts the feedback into data suitable for analysis.

Server decodes the payload, stores the feedback in persistent storage with an association to the corresponding case, and extracts the rating and comment text into internal data structures for further processing.

Input: feedback payload containing rating and comment.

Output: stored feedback records and in-memory representations of feedback data.

### Step 20

Server generates a feedback-analysis prompt sentence for the generative AI model and sends it for interpretation.

Server constructs a prompt sentence that includes the user's feedback text and explicit instructions to determine appropriateness, emotion, and keywords, and then transmits this prompt to the generative AI model for processing.

Input: feedback text and a feedback-analysis template.

Output: a feedback-analysis prompt sentence sent to the generative AI model.

### Step 21

Server receives and parses the feedback analysis result from the generative AI model.

Server obtains the generated text, identifies fields describing appropriateness, dominant emotion, and extracted keywords, and maps them into structured labels that can be stored and used for learning.

Input: generated feedback-analysis text.

Output: structured feedback analysis data including appropriateness labels, emotion labels, and key topics.

### Step 22

Server updates one or more model parameters or algorithm settings based on the structured feedback analysis data.

Server uses the feedback labels as training signals, aggregates them with corresponding feature data from past cases, and performs parameter updates in the urgency-prediction network or in prompt-generation heuristics to reduce future prediction error and improve recommendation alignment with user satisfaction.

Input: structured feedback analysis data and stored feature data for prior cases.

Output: updated model weights or configuration parameters that modify future behavior of the urgency-calculation and recommendation process.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Moreover, although the processing by the data processing system 10 described above was executed by the specific processing unit 290 of the data processing device 12 or by the control unit 46A of the smart device 14, the processing may be executed by a specific processing unit 290 of the data processing device 12 and a control unit 46A of the smart device 14. Moreover, the specific processing unit 290 of the data processing device 12 acquires and collects information needed for processing from the smart device 14 or from an external device or the like, and the smart device 14 acquires and collects information needed for processing from the data processing device 12 or from an external device or the like.

For example, a collection unit is implemented by the control unit 46A of the smart device 14 and/or by the specific processing unit 290 of the data processing device 12. For example, an acquisition unit acquires number-of-steps data using the camera 42 and/or the communication I/F 44 of the smart device 14, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the output device 40 of the smart device 14 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart device 14.

### Second Exemplary Embodiment

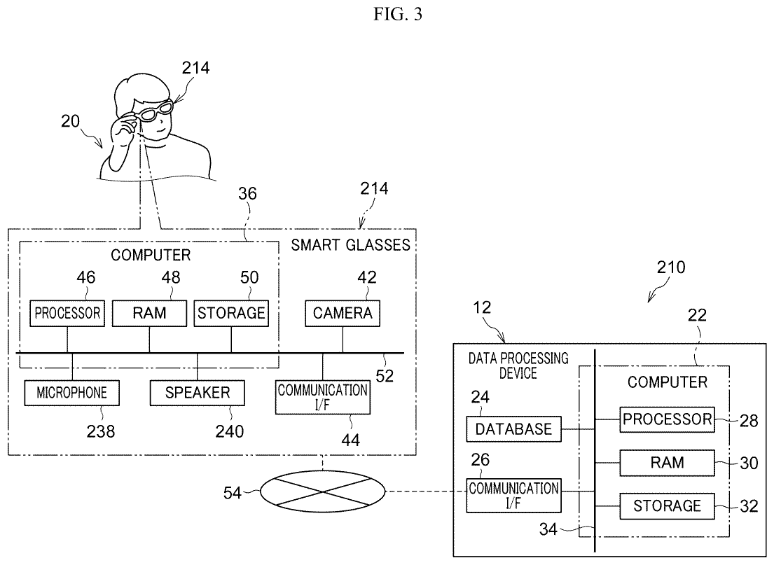

FIG. 3 illustrates an example of a configuration of a data processing system 210 according to a second exemplary embodiment.

As illustrated in FIG. 3, the data processing system 210 includes a data processing device 12 and smart glasses 214. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart glasses 214 include a computer 36, a microphone 238, a speaker 240, a camera 42, and a communication I/F 44. The computer 36 includes a processor 46, RAM 48, and storage 50. The processor 46, the RAM 48, and the storage 50 are connected to a bus 52. The microphone 238, the speaker 240, the camera 42, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

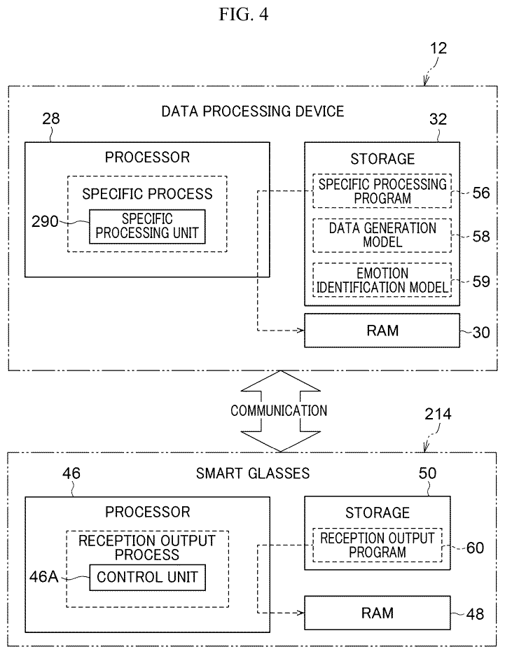

FIG. 4 illustrates an example of relevant functions of the data processing device 12 and the smart glasses 214. As illustrated in FIG. 4, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart glasses 214. A reception and output program 60 is stored in the storage 50. The processor 46 reads the reception and output program 60 from the storage 50 and in the RAM 48 executes the read reception and output program 60. The reception and output processing is implemented by the processor 46 operating as the control unit 46A according to the reception and output program 60 executed in the RAM 48. Note that a configuration may be adopted in which the smart glasses 214 include a data generation model and an emotion identification model similar to the data generation model 58 and the emotion identification model 59, and processing similar to the specific processing unit 290 is performed using these models.

Next, description follows regarding the specific processing by the specific processing unit 290 of the data processing device 12. The units of the system described below are implemented by the data processing device 12 and the smart glasses 214. In the following description the data processing device 12 is called a “server”, and the smart glasses 214 is called a “terminal”.

### Example 1

Explanation of flow will be omitted due to being similar to a flow of the specific processing in Example 1 as described in the first exemplary embodiment above.

Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

Application Example 2

Explanation of flow will be omitted due to being similar to a flow of the specific processing in Application Example 2 as described in the first exemplary embodiment above.

The specific processing unit 290 transmits a result of the specific processing to the smart glasses 214. The control unit 46A in the smart glasses 214 outputs the specific processing result to the speaker 240. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the smart glasses 214, the processing may be executed by a specific processing unit 290 of the data processing device 12 and a control unit 46A of the smart glasses 214. Moreover, the specific processing unit 290 of the data processing device 12 acquires and collects information needed for processing from the smart glasses 214 or from an external device or the like, and the smart glasses 214 acquires and collects information needed for processing from the data processing device 12 or from an external device or the like.

For example, the collection unit is implemented by the control unit 46A of the smart glasses 214 and/or by the specific processing unit 290 of the data processing device 12. For example, an acquisition unit acquires number-of-steps data using the camera 42 and/or the communication I/F 44 of the smart glasses 214, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 of the smart glasses 214 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart glasses 214.

### Third Exemplary Embodiment

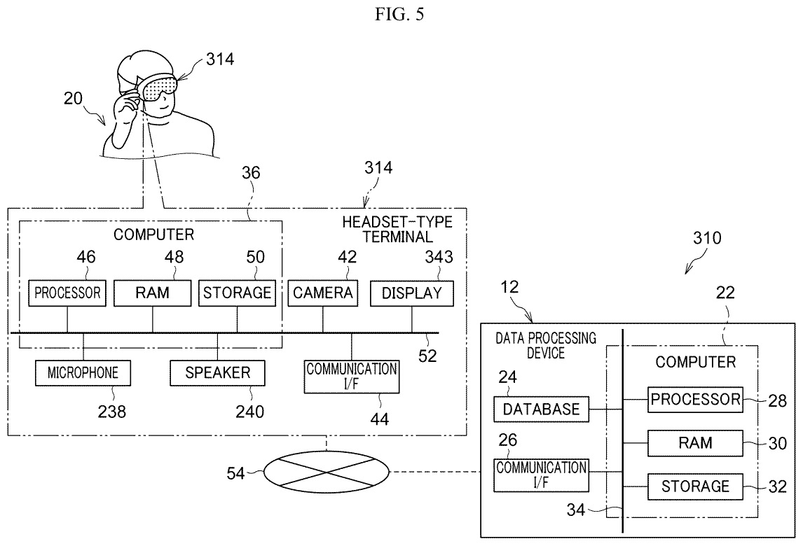

FIG. 5 illustrates an example of a configuration of a data processing system 310 according to a third exemplary embodiment.

As illustrated in FIG. 5, the data processing system 310 includes a data processing device 12 and a headset-type terminal 314. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The headset-type terminal 314 includes a computer 36, a microphone 238, a speaker 240, a camera 42, a communication I/F 44, and a display 343. The computer 36 includes a processor 46, RAM 48, and storage 50. The processor 46, the RAM 48, and the storage 50 are connected to a bus 52. The microphone 238, the speaker 240, the camera 42, the display 343, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

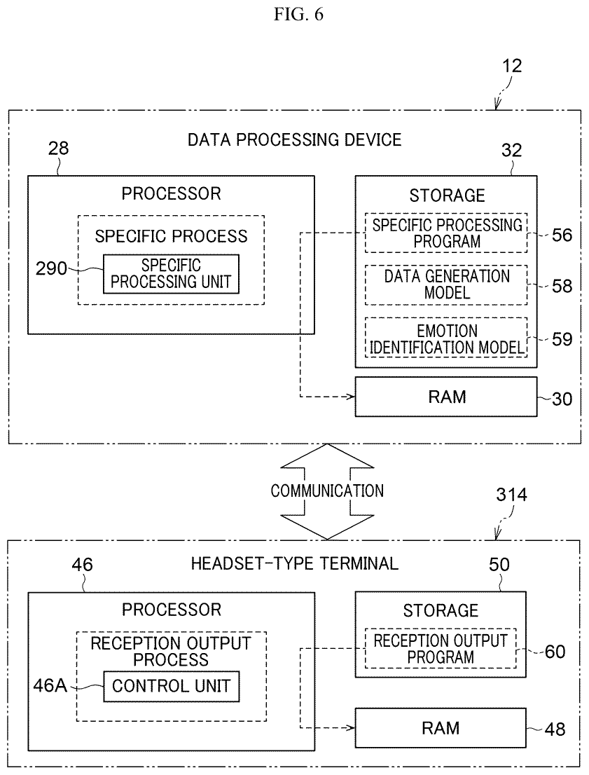

FIG. 6 illustrates an example of relevant functions of the data processing device 12 and the headset-type terminal 314. As illustrated in FIG. 6, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the headset-type terminal 314. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the headset-type terminal 314. In the following description the data processing device 12 is called a “server”, and the headset-type terminal 314 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the headset-type terminal 314. In the headset-type terminal 314, the control unit 46A outputs the result of the specific processing to the speaker 240 and the display 343. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the headset-type terminal 314, the processing may be executed by a specific processing unit 290 of the data processing device 12 and a control unit 46A of the headset-type terminal 314. Moreover, the specific processing unit 290 of the data processing device 12 acquires and collects information needed for processing from the headset-type terminal 314 or from an external device or the like, and the headset-type terminal 314 acquires and collects information needed for processing from the data processing device 12 or from an external device or the like.

For example, the collection unit is implemented by the control unit 46A of the headset-type terminal 314 and/or by the specific processing unit 290 of the data processing device 12. For example, an acquisition unit acquires number-of-steps data using the camera 42 and/or the communication I/F 44 of the headset-type terminal 314, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the display 343 of the headset-type terminal 314 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the headset-type terminal 314.

### Fourth Exemplary Embodiment

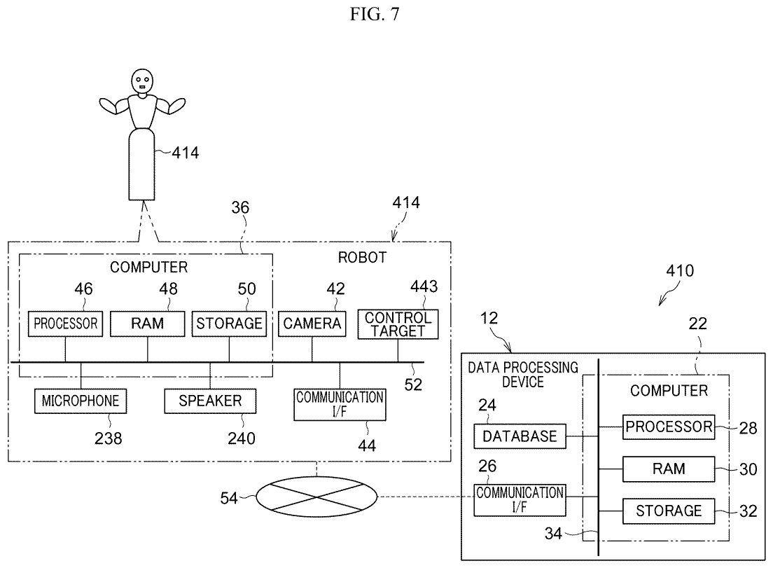

FIG. 7 illustrates an example of a configuration of a data processing system 410 according to a fourth exemplary embodiment

As illustrated in FIG. 7, the data processing system 410 includes a data processing device 12 and a robot 414. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The robot 414 includes a computer 36, a microphone 238, a speaker 240, a camera 42, a communication I/F 44, and a control target 443. The computer 36 includes a processor 46, RAM 48, and storage 50. The processor 46, the RAM 48, and the storage 50 are connected to a bus 52. The microphone 238, the speaker 240, the camera 42, the control target 443, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the robot 414 (for example, with an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

The control target 443 includes a display device, eye LEDs, and motors to drive arms, hands, feet, and the like. The posture and gesture of the robot 414 are controlled by controlling the motors of the arms, hands, feet, and the like. Part of an emotion of the robot 414 can be expressed by controlling these motors. Moreover, a facial expression of the robot 414 can be represented by controlling an illumination state of the eye LEDs of the robot 414.

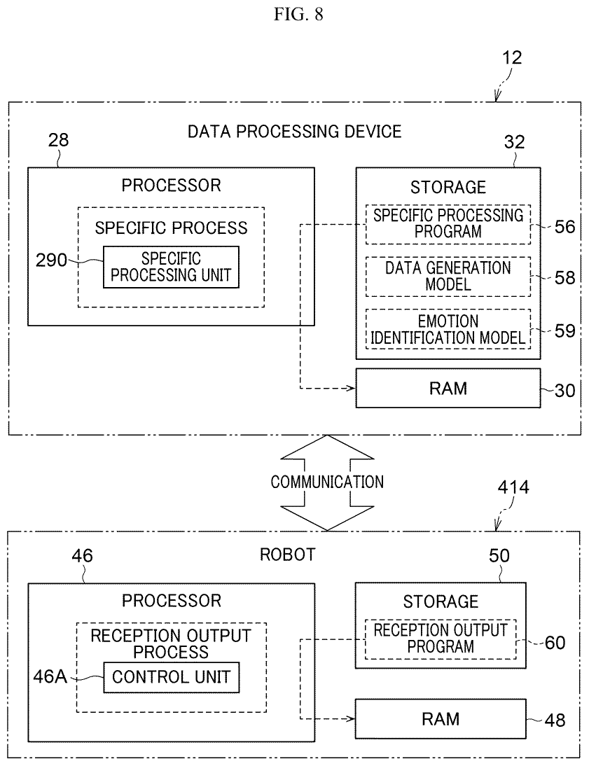

FIG. 8 illustrates an example of relevant functions of the data processing device 12 and the robot 414. As illustrated in FIG. 8, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the robot 414. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the robot 414. In the following description the data processing device 12 is called a “server”, and the robot 414 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the robot 414. In the robot 414, the control unit 46A outputs the result of the specific processing to the speaker 240 and the control target 443. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the robot 414, the processing may be executed by a specific processing unit 290 of the data processing device 12 and a control unit 46A of the robot 414. Moreover, the specific processing unit 290 of the data processing device 12 acquires and collects information needed for processing from the robot 414 or from an external device or the like, and the robot 414 acquires and collects information needed for processing from the data processing device 12 or from an external device or the like.

For example, the collection unit is implemented by the control unit 46A of the robot 414 and/or by the specific processing unit 290 of the data processing device 12. For example, an acquisition unit acquires number-of-steps data using the camera 42 and/or the communication I/F 44 of the robot 414, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the control target 443 of the robot 414 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the robot 414.

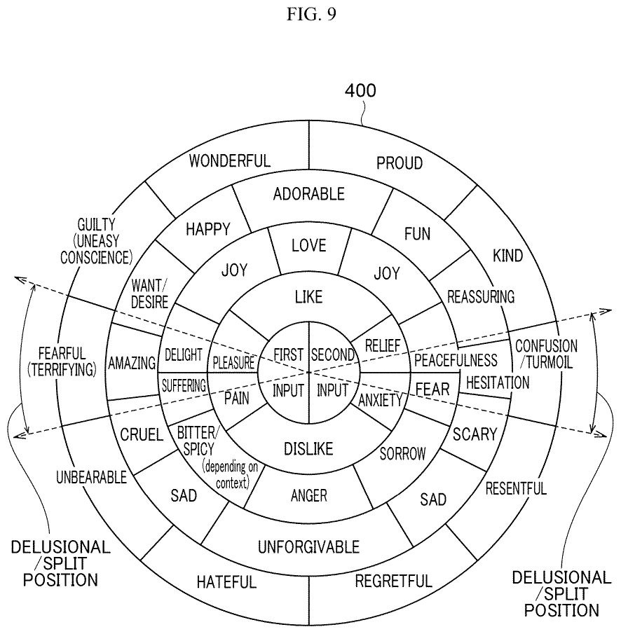

Note that the emotion identification model 59 serves as an emotion engine, and may decide the emotion of a user according to a specific mapping. Specifically, the emotion identification model 59 may decide the emotion of a user according to an emotion map (see FIG. 9) that is a specific mapping. Moreover, the emotion identification model 59 may also decide the emotion of the robot similarly, and the specific processing unit 290 may be configured so as to perform the specific processing using the emotion of the robot.

FIG. 9 is a diagram illustrating an emotion map 400 mapping plural emotions. In the emotion map 400, emotions are arranged in concentric circles that radiate out from the center. Primitive states of emotion are arranged nearer to the center of the concentric circles. Emotions expressing states and actions generated from states of mind are arranged further toward the outside of the concentric circles. Emotions are defined as including both affect and mental states. Emotions generated from reactions occurring in the brain are generally arranged at the left side of the concentric circles. Emotions induced by situational assessment are generally arranged at the right side of the concentric circles. Emotions generated from reactions occurring in the brain that are also emotions induced by situational assessment are generally arranged toward the top and toward the bottom of the concentric circles. Moreover, emotions of “euphoria” are arranged at the upper side of the concentric circles, and emotions of “dysphoria” are arranged at the lower side of the concentric circles. Plural emotions are accordingly mapped in this manner in the emotion map 400 based on a structure giving rise to emotions, and emotions that readily occur at the same time are mapped close to each other.

An example of such emotions is a distribution of emotions in the direction of 3 o'clock on the emotion map 400, generally around a boundary between relief and anxiety. Situational awareness dominates over internal sensations in the right half of the emotion map 400, with an impression of calm.

The inside of the emotion map 400 represents feelings, and the outside of the emotion map 400 represents actions, and so emotions further toward the outside of the emotion map 400 are more visible (are expressed by actions).

Human emotions are based on various balances, such as posture and blood sugar value balances, with a state of dysphoria being exhibited when these balances are far from ideal and a state of euphoria being exhibited when these balances are near to ideal. Even in a robot, a car, a motorbike, or the like, emotions can be thought of as being based on various balances such as orientation and remaining battery balances, with a state called dysphoria being exhibited when these balances are far from ideal and a state called euphoria being exhibited when these balances are near to ideal. An emotion map may, for example, be generated based on the emotion map of Dr. Mitsuyoshi (PhD Dissertation https://ci.nii.ac.jp/naid/500000375379: “Research on the phonetic recognition of feelings and a system for emotional physiological brain signal analysis”, Tokushima University). Emotions belonging to an area called “reaction” where feeling dominates are arranged in the left half of the emotion map. Moreover, emotions belonging to an area called “situation” where situational awareness dominates are arranged in the right half of the emotion map.

There are two types of emotion that facilitate leaning in an emotion map. One is an emotion in the vicinity of the center of negative “penitence” and “reflection” on the situational side. In other words, sometimes a negative “emotion” such as “I don't want to feel this way ever again” and “I don't want to be chided again” is experienced in a robot. Another is a positive emotion in the area of “desire” on the reaction side. In other words, there are times when a positive feeling such as “desire more” and “want to know more” is experienced.

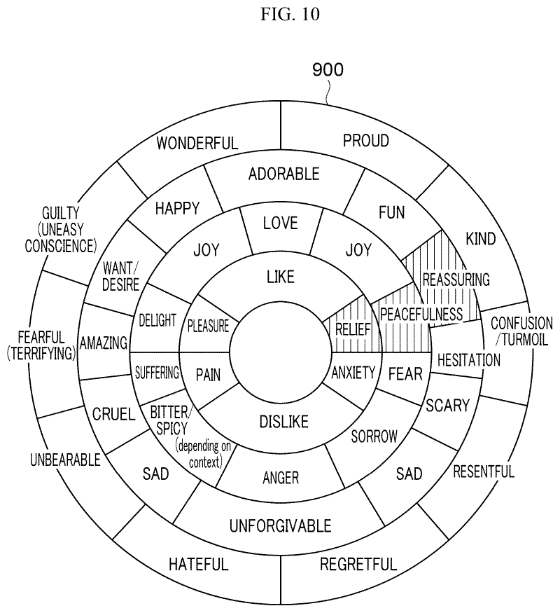

In the emotion identification model 59, user input is input to a pre-trained neural network, and emotion values indicating emotions shown on the emotion map 400 are acquired and the emotions of the user are decided. This neural network is pre-trained based on plural training data sets that each combine a user input with an emotion value indicating an emotion shown on the emotion map 400. The neural network is also trained such that emotions arranged close to each other have values that are close to each other, as in an emotion map 900 illustrated in FIG. 10. In FIG. 10 the plural emotions of “relief”, “peaceful”, and “reassured” are indicated as an example of close emotion values.

Although the system according to the present disclosure has been described mainly as functions of the data processing device 12, the system according to the present disclosure is not limited to being implemented in a server. The system according to the present disclosure may be implemented as a general information processing system. The present disclosure may, for example, be implemented by a software program operating on a personal computer, and may be implemented by an application operating on a smartphone or the like. The method according to the present disclosure may also be supplied to a user in the form of Software as a Service (SaaS).

Although in the exemplary embodiments described above examples are given of embodiments in which the specific processing is performed by a single computer 22, technology disclosed herein is not limited thereto, and distributed processing may be performed for the specific processing, with the specific processing distributed across plural computers including the computer 22. For example, the data generation model 58 may be provided in a device external to the data processing device 12, such that data generation in response to input data is performed in the external device.

Although in the exemplary embodiments described above examples are described of embodiments in which the specific processing program 56 is stored in the storage 32, the technology disclosed herein is not limited thereto. For example, the specific processing program 56 may be stored on a portable, non-transitory, computer readable, storage medium, such as universal serial bus (USB) memory or the like. The specific processing program 56 stored on the non-transitory storage medium is then installed on the computer 22 of the data processing device 12. The processor 28 then executes the specific processing according to the specific processing program 56.

Moreover, the specific processing program 56 may be stored on a storage device, such as a server connected to the data processing device 12 over the network 54, with the specific processing program 56 then being downloaded in response to a request from the data processing device 12 and installed on the computer 22.

Note that there is no need to store the entire specific processing program 56 on the storage device, such as a server connected to the data processing device 12 over the network 54, or to store the entire specific processing program 56 on the storage 32, and part of the specific processing program 56 may be stored thereon.

Hardware resources for executing the specific processing may use various processors as listed below. Examples of processors include, for example, a CPU that is a general-purpose processor that functions as a hardware resource to execute the specific processing by executing software, namely a program. Moreover, the processor may, for example, be a dedicated electronic circuit that is a processor having a circuit configuration custom designed for executing the specific processing, such as a field-programmable gate array (FPGA), a programmable logic device (PLD), or an application specific integrated circuit (ASIC). Memory is inbuilt or connected to each of these processors, and the specific processing is executed by each of these processors using the memory.

The hardware resource that executes the specific processing may be configured from one of these various processors, or may be configured from a combination of two or more processors of the same or different type (for example, a combination of plural FPGAs, or a combination of a CPU and a FPGA). The hardware resource executing the specific processing may be a single processor.

Examples of configurations of a single processor include, firstly, a configuration of a single processor resulting from combining one or more CPU and software, in an embodiment in which this processor functions as the hardware resource for executing the specific processing. Secondly, as typified by a System-on-chip (SOC) or the like, there is also an embodiment that uses a processor realized by a single IC chip to function as an overall system including plural hardware resources for executing the specific processing. Adopting such an approach means that the specific processing is realized using one or more of the various processors described above as hardware resource.

Furthermore, more specifically, an electrical circuit that combines circuit elements such as semiconductor elements or the like may be employed as a hardware structure of these various processors. The specific processing is merely an example thereof. This means that obviously redundant steps may be omitted, new steps may be added, and the processing sequence may be swapped around within a range not departing from the spirit of the present disclosure.

The described content and drawing content illustrated above are a detailed description of parts according to the present disclosure, and are merely examples of the present disclosure. For example, description related to the above configuration, function, operation, and advantageous effects is a description related to examples of the configuration, function, operation, and advantageous effects of parts according to the present disclosure. This means that obviously redundant parts may be eliminated, new elements may be added, and switching around may be performed on the described content and drawing content illustrated above within a range not departing from the spirit of the present disclosure. Moreover, to avoid misunderstanding and to facilitate understanding of parts according to the present disclosure, description related to common knowledge in the art and the like not particularly needing description to enable implementation of the present disclosure is omitted in the described content and drawing content illustrated as described above.

All publications, patent applications and technical standards mentioned in the present specification are incorporated by reference in the present specification to the same extent as if each individual publication, patent application, or technical standard was specifically and individually indicated to be incorporated by reference.

Note that, regarding the above description, the following supplementary notes are further disclosed.

### Example 1

### Supplementary 1

A system comprising a processor,
wherein the processor is configured to
cause a user terminal to present, on a display device, an information input screen as a user interface and to receive, from a user, a plurality of types of state information including cause information, symptom information, and elapsed-time information related to a health condition,
receive, via a communication device, the state information transmitted from the user terminal, convert the state information into structured data, store the structured data in a storage device, and preprocess the structured data to generate numerical feature data,
input the numerical feature data into a machine learning model to estimate disease-type information and injury-severity information, and calculate a visit-urgency index on the basis of the disease-type information, the injury-severity information, the elapsed-time information, and predetermined weighting parameters stored in the storage device,
generate, on the basis of the disease-type information, the injury-severity information, and the visit-urgency index, a prompt sentence that instructs a generative AI model to perform analysis and explanation generation, input the prompt sentence into the generative AI model, and obtain analysis-result information including explanation information in natural language understandable to the user and recommended medical-action information, and
cause the user terminal to visually present, in the user interface, the visit-urgency index and the analysis-result information so as to support an action selection by the user.

### Supplementary 2

The system according to supplementary 1,
wherein the processor is configured to
acquire, via the user interface of the user terminal, feedback information including at least one of evaluation information by the user with respect to the analysis-result information and the recommended medical-action information, and an actual action-history information of the user, generate a prompt sentence that instructs the machine learning model and the generative AI model to perform analysis on the basis of a state-information history including the feedback information, and improve accuracy of the visit-urgency index and the analysis-result information by performing at least one of adjusting parameters of the machine learning model and updating the weighting parameters.

### Supplementary 3

The system according to supplementary 1,
wherein the processor is configured to
in a case where the visit-urgency index is equal to or greater than a predetermined threshold and the injury-severity information is estimated as high risk, generate a prompt sentence that instructs the generative AI model to generate an emergency-contact text including summary information of contact content to be transmitted to an emergency responder and user state information, and automatically transmit the emergency-contact text to an emergency-response device via the communication device.

### Application Example 1

### Supplementary 1

A system comprising a processor,
wherein the processor is configured to
provide a user interface on an information processing device to receive health-related information including at least a cause, a symptom, and an elapsed time,
normalize the received health-related information and extract feature data from the normalized information,
execute, on the information processing device, a machine learning model using the feature data to estimate a disease name and a severity level of a health condition,
generate a prompt sentence, on the information processing device, based on at least the received health-related information and the estimated disease name and severity level, the prompt sentence instructing a generative AI model to perform an analysis,
transmit the generated prompt sentence to an external generative AI model and receive, from the external generative AI model, an analysis result including at least an explanatory text and recommended response content,
calculate, on the information processing device, an urgency of visiting a medical institution as a numerical index, based on the estimated disease name and severity level and on the analysis result received from the external generative AI model, the calculation including applying a weighting according to the disease name and severity level and a confidence value,
present, via the user interface, the calculated urgency of visiting the medical institution, the disease name, the severity level, and the recommended response content included in the analysis result, and
store, in a storage medium, the received health-related information, the estimated disease name and severity level, and the analysis result, and update at least one of the machine learning model and a generation rule of the prompt sentence based on stored data used as learning data.

### Supplementary 2

The system according to supplementary 1,
wherein the processor is configured to
acquire, through the user interface, feedback information including at least an evaluation or correction from a user, regenerate the prompt sentence for the generative AI model based on the feedback information and the health-related information, analyze the feedback information, and modify at least one of a weighting of the machine learning model and a structure of the prompt sentence so as to improve an accuracy of the system.

### Supplementary 3

The system according to supplementary 1,
wherein the processor is configured to
when the calculated urgency of visiting the medical institution exceeds a predetermined threshold, generate a prompt sentence for the generative AI model that requests generation of contact content including at least emergency contact information and current symptom information, transmit the prompt sentence to the generative AI model, receive the contact content generated by the generative AI model, and automatically transmit the contact content to an emergency destination via a communication function.

### Example 2

### Supplementary 1

A system comprising a processor, a storage device, an information input/output device, and a communication device,
wherein the processor is configured to
acquire, via the information input/output device, input information from a user including state information comprising at least a cause, a symptom, and an elapsed time, and store the acquired input information as structured analysis data in the storage device; and
generate, based on the structured analysis data stored in the storage device, a prompt sentence in which evaluation target information including the state information is embedded in a natural language format and/or a structured data format, supply the generated prompt sentence to a generative AI model, and obtain from the generative AI model an analysis result including at least a health state, an abnormality severity, and an emergency level; and
calculate, based on numerical information and/or classification information included in the analysis result, an evaluation index indicating a consultation urgency, and present the evaluation index and recommendation information obtained from the generative AI model to the user via the information input/output device; and
store, in the storage device, the structured analysis data, the analysis result, and the evaluation index in association with one another as history information that is referable in subsequent analysis processing.

### Supplementary 2

The system according to supplementary 1,
wherein the processor is configured to
receive, via the information input/output device, feedback information from the user including evaluation information regarding at least one of the recommendation information and the consultation urgency, and store the feedback information in the storage device in association with the history information; and
acquire, from the storage device, a plurality of case pieces of information each including a combination of the input information, the analysis result, and the feedback information, generate an update prompt sentence that describes, in a natural language format, a learning context including the plurality of case pieces of information, and supply the update prompt sentence to the generative AI model to obtain a model response indicating update content of evaluation parameters and/or decision rules for calculation of the consultation urgency and generation of the recommendation information; and
update, based on the update content included in the model response, threshold values, weighting coefficients, classification boundaries, or other control parameters used in consultation urgency calculation processing and recommendation information generation processing, as setting information in the storage device, and execute subsequent analysis processing for new input information using the updated setting information.

### Supplementary 3

The system according to supplementary 1,
wherein the processor is configured to
determine, based on at least one of the analysis result obtained from the generative AI model and the updated setting information, that the consultation urgency is equal to or higher than a predetermined reference value, generate a contact-sentence generation prompt sentence for generating contact content to an emergency response organization, and supply the contact-sentence generation prompt sentence to the generative AI model to obtain contact sentence information to be transmitted to the emergency response organization; and
generate notification data including the contact sentence information and user identification information, transmit the notification data to an external emergency response organization via the communication device, and store a transmission history of the notification data in the storage device.

### Application Example 2

### Supplementary 1

A system comprising a processor,
wherein the processor is configured to
provide a user interface that receives situation information including at least a cause, a symptom, and an elapsed time from a terminal device used by a user,
acquire biometric information and emotion information from the terminal device, the biometric information including at least vital sign data and the emotion information indicating a psychological state of the user,
perform preprocessing on the situation information, the biometric information, and the emotion information to convert the situation information, the biometric information, and the emotion information into feature data,
generate a prompt sentence that instructs a generative AI model to perform analysis using the feature data,
input the prompt sentence into the generative AI model and obtain an analysis result from the generative AI model,
calculate, on the basis of the analysis result and the feature data, a visit urgency or an emergency index indicating a necessity of medical visitation,
generate response content including at least a recommendation for an action according to the visit urgency or the emergency index, and cause the user interface to display the response content, and
when the visit urgency or the emergency index exceeds a predetermined threshold, automatically connect to an emergency contact via a communication function.

### Supplementary 2

The system according to supplementary 1,
wherein the processor is configured to
acquire evaluation information including feedback from the user,
generate a prompt sentence that instructs the generative AI model to analyze the evaluation information to extract at least an emotion state and an appropriateness of a past recommendation, and input the prompt sentence into the generative AI model to obtain a feedback analysis result, and
update one or more parameters of at least one of the generative AI model and an algorithm for calculating the visit urgency or the emergency index, on the basis of the feedback analysis result, so as to improve recommendation accuracy and user satisfaction.

### Supplementary 3

The system according to supplementary 1,
wherein the processor is configured to
generate a prompt sentence that instructs the generative AI model to generate emergency contact content, on the basis of the feature data and the analysis result, and input the prompt sentence into the generative AI model to obtain the emergency contact content, and
create notification data including at least the emergency contact content, location information, and the biometric information, and automatically transmit the notification data to an emergency response organization via the communication function.

What is claimed is:

1. A system comprising:
circuitry configured to:
receive, via a communication interface coupled to a packet-switched network, input data from a terminal device, the input data comprising a plurality of data fields;
convert the input data into structured feature data by applying a preprocessing operation to the plurality of data fields;
generate a prompt data sequence based on the structured feature data, the prompt data sequence being configured to instruct a generative neural network model to perform an inference operation;
input the prompt data sequence into the generative neural network model and obtain inference result data from the generative neural network model;
compute an evaluation index based on at least the inference result data and the structured feature data; and
transmit, via the communication interface, response data comprising the evaluation index and at least a portion of the inference result data to the terminal device for presentation on a display of the terminal device.

2. The system according to claim 1, wherein the circuitry is further configured to:
store the structured feature data, the inference result data, and the evaluation index in a storage device as associated history data; and
retrieve, from the storage device, the associated history data for use in a subsequent inference operation.

3. The system according to claim 2, wherein the circuitry is further configured to:
receive, via the communication interface, feedback data from the terminal device;
store the feedback data in the storage device in association with the history data;
acquire a plurality of case records from the storage device, each case record comprising a combination of the input data, the inference result data, and the feedback data; and
generate an update prompt data sequence that describes, in a natural language format, a learning context comprising the plurality of case records.

4. The system according to claim 3, wherein the circuitry is further configured to:
supply the update prompt data sequence to the generative neural network model to obtain a model response indicating update content of evaluation parameters; and
update, based on the update content, threshold values, weighting coefficients, or classification boundaries used in computation of the evaluation index, as setting information in the storage device.

5. The system according to claim 4, wherein the feedback data comprises at least one of evaluation information regarding a recommendation provided to a user and action-history information indicating an actual action taken by the user after receiving the recommendation, and wherein the update content adjusts the weighting coefficients to reduce a discrepancy between the evaluation index and the action-history information.

6. The system according to claim 1, wherein the converting the input data into structured feature data comprises:
tokenizing text portions of the input data into a token sequence;
mapping each token of the token sequence to a numerical index using a vocabulary stored in a storage device; and
computing a numerical vector representation based on the mapped numerical indices.

7. The system according to claim 6, wherein the preprocessing operation further comprises:
converting a temporal data field of the plurality of data fields into a standardized numerical duration value; and
normalizing the standardized numerical duration value using a scaling parameter stored in the storage device,
wherein the structured feature data comprises a concatenation of the numerical vector representation and the normalized numerical duration value.

8. The system according to claim 7, wherein the circuitry is further configured to:
input the structured feature data into a machine learning classification model stored in the storage device;
obtain, from the machine learning classification model, category information and a severity indicator; and
wherein the evaluation index is computed as a weighted combination of the category information, the severity indicator, and the normalized numerical duration value using predetermined weighting parameters stored in the storage device.

9. The system according to claim 8, wherein the category information comprises disease-type information indicating one or more candidate categories of a health condition, and the severity indicator comprises an injury-severity score, and wherein the evaluation index represents a visit-urgency index indicating a degree of urgency for visiting a medical institution.

10. The system according to claim 9, wherein the prompt data sequence is generated by embedding the disease-type information, the injury-severity score, and the visit-urgency index into a natural language template, the prompt data sequence instructing the generative neural network model to generate explanation information in natural language understandable to a user and recommended medical-action information.

11. The system according to claim 1, wherein the circuitry is further configured to:
determine whether the evaluation index satisfies a predetermined threshold condition; and
in response to the evaluation index satisfying the predetermined threshold condition, generate a notification prompt data sequence instructing the generative neural network model to generate notification content.

12. The system according to claim 11, wherein the circuitry is further configured to:
input the notification prompt data sequence into the generative neural network model to obtain notification text data;
generate notification data comprising the notification text data and identification information associated with a user of the terminal device; and
transmit the notification data to an external device via the communication interface.

13. The system according to claim 12, wherein the external device is a device associated with an emergency response organization, and wherein the notification text data comprises a summary of a user condition, a severity assessment, and a recommended response action generated by the generative neural network model.

14. The system according to claim 13, wherein the circuitry is further configured to:
store a transmission history of the notification data in a storage device, the transmission history comprising at least a destination identifier, a transmission timestamp, and a content summary; and
associate the transmission history with the history data for subsequent reference.

15. The system according to claim 1, wherein the input data further comprises biometric sensor data acquired from a sensor coupled to the terminal device and emotion indicator data derived from at least one of audio data and image data captured by the terminal device, and wherein the structured feature data is generated by preprocessing the plurality of data fields, the biometric sensor data, and the emotion indicator data into a unified numerical feature representation.

16. The system according to claim 15, wherein the biometric sensor data comprises at least one of a heart rate value, a blood pressure value, and a body temperature value, and wherein the emotion indicator data comprises an emotion classification label derived from an emotion identification model stored in a storage device.

17. The system according to claim 1, wherein the generative neural network model comprises a transformer-based architecture including an embedding layer, a plurality of self-attention layers, and an output layer configured to generate a sequence of output tokens based on the prompt data sequence.

18. A system comprising:
circuitry configured to:
receive, via a communication interface coupled to a packet-switched network, input data from a terminal device, the input data comprising a plurality of data fields including at least a cause data field, a symptom data field, and a temporal data field;
convert the input data into structured feature data by tokenizing text portions of the input data, mapping tokens to numerical indices using a stored vocabulary, computing a numerical vector representation, converting the temporal data field into a standardized numerical duration value, and concatenating the numerical vector representation and the standardized numerical duration value;
input the structured feature data into a machine learning classification model to obtain category information and a severity indicator;
compute an evaluation index as a weighted combination of the category information, the severity indicator, and the standardized numerical duration value using predetermined weighting parameters;
generate a prompt data sequence by embedding the category information, the severity indicator, and the evaluation index into a natural language template, the prompt data sequence being configured to instruct a generative neural network model comprising a transformer-based architecture to perform an inference operation;
input the prompt data sequence into the generative neural network model and obtain inference result data comprising explanation text data and recommended action text data; and
transmit, via the communication interface, response data comprising the evaluation index and the inference result data to the terminal device for presentation on a display of the terminal device.

19. The system according to claim 18, wherein the circuitry is further configured to:
determine whether the evaluation index exceeds a predetermined threshold;
in response to the evaluation index exceeding the predetermined threshold, generate a notification prompt data sequence instructing the generative neural network model to generate emergency contact text data;
input the notification prompt data sequence into the generative neural network model to obtain the emergency contact text data;
generate notification data comprising the emergency contact text data and user identification information; and
transmit the notification data to a device associated with an emergency response organization via the communication interface.

20. A method comprising:
receiving, via a communication interface coupled to a packet-switched network, input data from a terminal device, the input data comprising a plurality of data fields;
converting the input data into structured feature data by applying a preprocessing operation to the plurality of data fields;
generating a prompt data sequence based on the structured feature data, the prompt data sequence being configured to instruct a generative neural network model to perform an inference operation;
inputting the prompt data sequence into the generative neural network model and obtaining inference result data from the generative neural network model;
computing an evaluation index based on at least the inference result data and the structured feature data; and
transmitting, via the communication interface, response data comprising the evaluation index and at least a portion of the inference result data to the terminal device for presentation on a display of the terminal device.
